(19) United States
(12) Patent Application Publication
(10) Pub. No.: US 2020/0000477 A1
(43) Pub. Date: Jan. 2, 2020

(54) Embolization Plug

(71) Applicant: Henry Nita LLC, Redwood City, CA (US)

(72) Inventors: Henry Nita, Redwood City, CA (US); Jeff Sarge, Fremont, CA (US)

(21) Appl. No.: 16/566,521

(22) Filed: Sep. 10, 2019

Related U.S. Application Data
(63) Continuation-in-part of application No. 16/031,848, filed on Jul. 10, 2018.
Application No. 16/031,848 is a continuation-in-part of application No. 15/947,842, filed on Apr. 8, 2018.
Application No. 15/947,842 is a continuation-in-part of application No. 15/227,713, filed on Aug. 3, 2016, now abandoned.
(60) Provisional application No. 62/893,587, filed on Aug. 29, 2019.

(51) Int. Cl. A61B 17/12

(52) U.S. Cl. CPC A61B 17/12172; A61B 2017/00867; A61B 17/12163; A61B 17/12113

(57) ABSTRACT

Devices and methods that a variety of embolization plugs for treatment of endovascular and non-endovascular defects are disclosed. The plugs have multiple braids that include an outer expandable braid and an inner expandable braid that is located inside the outer expandable braid.

### FIELD OF THE INVENTION

The present invention relates to methods and devices to treat endovascular and non-endovascular defects including but not limited to parent vessel occlusion, cerebral and endovascular aneurysms, arterial-venous malformations, embolism or prevention of blood flow to tumors or other portions of the body. Treatment of other medical conditions including congenital defects such as Atrial and Ventricular Septal Defects, Patent Ductus Arteriosus, pre-SIRT Radiation Therapy (Gastroduodenal Artery Embolization), gastro-intestinal bleeding, Pulmonary AVM, Pre-EVAR (Endo Vascular Aneurysm Repair) Internal Iliac Embolization, Congestive Heart Disease and Patent Foramen Ovale are also included. The devices made in accordance with the invention are particularly well suited for delivery through a catheter or the like to a remote location in a patient's body.

### BACKGROUND OF THE INVENTION

The devices described in this invention are intended, among other therapies, for treatment of defects in the arteries and veins. The defects include aneurysms, fusiform aneurysms, arteriovenous malformations, arteriovenous fistulas, cavernous fistulas and dissections, as well as other hyper-vascular lesions such as head and neck tumors, etc. These defects cause a variety of symptoms, ranging from pain, weakness, headache, vision loss, and stroke to death. Preferably, these defects would be treated with devices and methods of the present invention that leave the associated parent artery or vein intact and patent, so it may continue to supply blood and function normally. However, in many cases, a patient's condition may dictate that immediate cessation of blood flow is required.

When parent artery preservation is not advisable, the devices and methods of the present invention can be used for parent artery occlusion (PAO). Parent artery occlusion is accomplished by quickly and securely closing off a length of a blood vessel near the defect that preferably results in immediate and complete blockage of blood flow to the defect, and permanent isolation of the blood vessel segment near the defect. Parent artery occlusion is sometimes referred to more broadly as parent vessel occlusion to encompass occlusion of both arteries and veins.

Several endovascular devices and techniques have been developed to accomplish parent artery occlusion. Detachable balloons have previously been used for parent artery occlusion but were not successful because of leakage and unexpected deflation, leading to major complications. Occlusive coils have been used to pack fusiform aneurysms and cavernous fistulas, but often do not result in immediate occlusion. As a result, trickling blood flow which occurs for several minutes while the patient's blood is coagulating around the mass of coils may lead to creation and migration of thrombus from the mass of coils.

Vascular plugs have also been used to accomplish parent artery occlusion. Currently available plugs such as the Amplatzer vascular plug are difficult to deploy and are size-sensitive. Also, the open-mesh construction of these vascular plugs may result in dislodgement of thrombus as it is forming on the plug, leading to downstream embolization of the occluded artery.

Mechanical embolization devices such as filters and traps have been proposed in the past to achieve parent artery occlusion and are disclosed in U.S. Pat. Nos. 3,874,388; 5,334,217; 4,917,089 and 5,108,420 among others, however, deployment of these devices and/or recapture into the delivery catheter is difficult, further limiting the effectiveness of these devices.

An aneurysm is an abnormal bulge or ballooning of the wall of a blood vessel, which most commonly occurs in arterial blood vessels. Aneurysms typically form at a weakened point of a wall of a blood vessel. The force of the blood pressure against the weakened wall causes the wall to abnormally bulge or balloon outside. Aneurysms, particularly cranial aneurysms, are a serious medical condition because they can apply undesired pressure to areas within the brain. Additionally, there is always the possibility that the aneurysm may rupture or burst leading to serious medical complications including death.

More recently, less invasive intravascular catheter techniques have been used to treat endovascular and cranial aneurysms. Typically, these techniques involve use of a catheter to deliver platinum coils, currently the most popular embolic devices, to a treatment area within the vasculature. In the case of a cranial aneurysm, a delivery catheter is inserted through a guiding catheter to the site of the cranial aneurysm. A platinum coil attached to the pusher wire is pushed through the delivery catheter, and into the aneurysm. Once platinum coils have been deployed within the aneurysm, blood clots (thrombus) are formed. Formation of such blood clots will seal off the aneurysm preventing further ballooning or rupture. The coil deployment procedure is repeated until the packing density within the aneurysm reaches about 30% or more of the volume.

There are a variety of materials and devices which have been used for treatment of vascular aneurysms, including platinum and stainless-steel coils, polyvinyl alcohol sponges, and other mechanical devices. One type of widely-used occlusion implant is helical wire coils described in U.S. Pat. Nos. 4,994,069 and 6,299,627. Occlusion coils having attached fibrous elements are disclosed in U.S. Pat. Nos. 5,833,705; 5,304,194; 5,354,295; 5,122,136 and describe electrolytically detachable occlusion implants. Occlusion coils having little or no inherent secondary shape have been described in U.S. Pat. Nos. 5,690,666; 5,826,587; and 6,458,119 while U.S. Pat. No. 5,382,259 describes non-expanding braids covering a primary coil structure.

Occlusion implant compositions comprising one or more expandable hydrogels have also been described in U.S. Pat. Nos. 6,960,617; 6,113,629; 6,602,261 and 6,238,403 which disclose a plurality of expansible hydrogel elements disposed at spaced intervals along a filamentous carrier. Other U.S. Pat. Nos. 6,616,617; 6,475,169; 6,168,570 and 6,159,165 disclose multi-stranded micro-cable devices, where one or more of the strands may be an expandable material. Occlusion implants made of a combination of braid with underlining coils that should serve as a blood diverter when deployed inside the aneurysm are described in U.S. Pat. Nos. 9,011,482 and 9,060,777.

Despite the above, a need remains for occlusion implants having a better packing capability and filling density, and preferably made of a single occlusive device suitable for multiple clinical applications, either for parent vessel occlusion, neurological or other endovascular aneurysm occlusion, or other defects in the human body.

### SUMMARY OF THE INVENTION

The devices and methods described in the present invention are suitable for parent artery occlusion within the human endovascular system, including cerebral arteries and veins, and may be used to treat aneurysms throughout the body.

The embolization devices of the present invention include detachable tandem embolization devices (TED), occlusion implants comprising at least one expandable braid and at least one coil; detachable mesh endo-frame devices (MEF), occlusion implants comprising at least one expandable braid with a constraining member inside the braid, and occlusion implants comprising dual braids or a braid-inside-braid structure, with or without an attached coil.

The tandem embolization devices (TED) or occlusion implants of the present invention comprise at least one elongate expandable braid and at least one coil. The occlusion implants are attached to a pusher member with a detachable electro-mechanical attachment means and positioned inside the delivery catheter. When released/detached from the pusher member and outside of the delivery catheter, the occlusion implant expands to its unrestrained shape and/or to the extent allowed by the surrounding treatment area. In one primary embodiment, deployment of a distal expandable braid(s) from the delivery catheter forms a pre-shaped anchoring structure that results in larger space coverage, while the attached coil(s) provides a final packing of the treatment area and immediate occlusion of the artery or aneurysm. In another primary embodiment; deployment of a distal coil from the delivery catheter forms a pre-shaped anchoring structure around the treatment area, while the attached expandable braid provides a final packing of the treatment area and immediate occlusion of the artery or aneurysm.

The occlusion implants of the present invention include at least one elongate expandable braid attached to a pusher member with a detachable mechanical attachment means and positioned inside the delivery catheter.

One objective of the present invention is to provide an occlusion implant that at least partially expands to occupy a greater volume within the treatment area than conventional helical coils, thus providing an effective engaging/anchoring edifice combined with a large volumetric area to promote quick blood clotting.

In one embodiment of the present invention, an occlusion device or system for occluding endovascular defects comprises a delivery catheter having a distal end and a proximal end, an elongate occlusion implant extending longitudinally within the delivery catheter and configured to be pushed through and out of the delivery catheter and retrieved back into the distal end of the delivery catheter using a pushing member. The occlusion implant comprises at least two regions: a first distal region comprised of an expandable braid element and a second elongate region proximal to the first distal braid and comprised of a non-expandable helical coil. Such a hybrid structure or tandem structure of a braid and a coil at least partially expands to a larger volumetric area when pushed out of delivery catheter. The expanded braid is configured to have a pre-set expanded longitudinal shape when released from the delivery catheter. The expanded braid may also have a bulbous shape resembling a bulb in shape, or rounded or swollen, as well as, any shape suitable to fill out a treatment area. The occlusion implant traverses concomitant bends as the delivery catheter when delivered through the delivery catheter to the treatment location.

In another embodiment, the expandable braid has a collapsed configuration when held inside the delivery catheter and an expanded configuration that is radially larger than the second elongate helical coil region when in a released configuration outside the delivery catheter.

In another embodiment, the braid is connected to the helical coil, and such braid and helical coil connections may be formed by one or more of the following methods; directly connected, using an intermediate member, and a combination of both. Such connection may be achieved by bonding, fusing, welding, soldering, gluing or other mechanical or thermal means.

In another embodiment, the helical coil may be wound from an extension of one or more of the braid strands, thereby making the braid and coil a continuous mechanical structure and thus eliminating the need for any additional bonded connection between the two.

In yet another embodiment, the braid of the occlusion implant has a longer length when in the collapsed configuration inside the delivery catheter than its actual length when deployed outside the delivery catheter.

In another embodiment, the braid has a formed distal tip wherein the braid strands are prolapsed back into the distal inside diameter of the braid, thereby minimizing delivery friction through the catheter, yet enhancing anchoring of the implant in the patient while minimizing the potential for vessel trauma during deployment.

In yet another embodiment, the braid has a formed distal tip that prevents the very distal section of the braid from fully expanding when deployed from the delivery catheter. Such a distal tip may be made of one of the following materials: metal, polymer, rubber, adhesive or a combination of thereof.

In another embodiment, at least one radiopaque marker is positioned along the occlusion implant including at any of the following locations: the distal end, the proximal end, along the length of the implant, or any combination thereof. A radiopaque marker may be positioned inside the occlusion implant, on the outside surface thereof, or on both locations. A radiopaque marker may include a radiopaque solder.

In yet another embodiment, the helical coil is attached proximally to a pushing member (pusher) located at least partially within the delivery catheter. The pushing member is constructed to push the occlusion implant out of the delivery catheter, deploy and retrieve the occlusion implant from and into the delivery catheter when needed.

In another embodiment, at least one elongate constraining member is extended at least partially through the helical coil, and it is attached to or near the distal end of the helical coil and to or near to the proximal end of the helical coil. Alternatively, or in addition, at least one elongate constraining member is extended through the occlusion implant and it is attached distally to or near the distal end of the braid and proximally to or near the proximal end of the helical coil.

In yet another embodiment, the elongate constraining member has variable stiffness along its length, being stiffer distally and more flexible proximally. Alternatively, the elongate constraining member has a variable flexibility along its length, with more flexibility distally and less flexibility proximally. The elongate constraining member can also have a more flexible proximal end and less flexible distal end.

In another embodiment, the elongate constraining member may enhance the thrombogenicity of the implant when deployed in endovascular or non-endovascular defects.

In yet another embodiment, the elongate constraining member may enhance the radiopacity of the occlusion implant by virtue of its composition.

In another embodiment, the braid comprises a proximally tapered section to facilitate deployment and retrieval of the braid from the delivery catheter.

In yet another embodiment, the helical coil has variable flexibility, being stiffer distally and more flexible proximally. Alternatively, the helical coil may be more flexible distally and less flexible proximally.

In yet another embodiment, the first braided region is made of a braid that has a diameter that is at least 1.3 times larger than the diameter of the second region helical coil when the occlusion implant is released from the delivery catheter.

In yet another embodiment, the braid is formed from a plurality of strands of Nitinol wire having an outside diameter between 0.0003 inches and 0.010 inches. The braided material is formed from a plurality of strands having a pore size formed between strands in the expanded configuration of less than about 0.2 square mm. The braid may be formed from a plurality of strands of Nitinol wire having multiple wire strands of the same dimensions or of different dimensions braided into the shape using a circular wire, oval wire, flat wire and any other suitable wire configuration, or combinations thereof.

In another embodiment, the expanded braid may be configured to have a pre-set expanded diameter having a cross-sectional (transverse) shape in the following configurations: tubular, circular shape, bulbous shape, onion-shape resembling onion or any other shape including but not limited to non-circular, for example, oval, flat, rectangular, tear-shaped, twist-shape and other suitable shapes.

In another embodiment, the occlusion implant is at least partially configured to have pre-set longitudinal shapes including a curved shape, three-dimensional shape, helical shape, non-linear, random shape and any non-linear shape.

In yet another embodiment, the distal braid is configured to assume a radial configuration that opposes the inside wall of the defect after deployment from the delivery catheter, thereby creating a radial frame. Such a radial frame may anchor in the wall to prevent the occlusion implant from being repositioned by blood flow while the proximal helical coil fills the defect space upon deployment from the delivery catheter.

In another embodiment, the first region braid has an open braid on the distal end.

The embolization plugs of the present invention include detachable multi-braid structures comprising multiple braids, and having at least two expandable braids. Such a multi-braid structure provides a desired plug density to achieve a quick occlusion of the treatment area. The plug is attached to a pusher member with a detachable electro-mechanical or mechanical attachment mechanism and is positioned inside the delivery catheter. When released and detached from the pusher member and outside of the delivery catheter, the plug expands to its unrestrained or expanded shape in the treatment area. The deployment of the multi-braid structured plug from the delivery catheter forms a pre-shaped anchoring edifice that prevents the plug from moving after deployment while providing a desirable plug occlusion density that stops blood flow at the deployment area.

In yet another embodiment, at least one radial elongate constraining member is positioned at least one location around and along the braid region.

In another embodiment, an alternative or additional friction reduction means are located within the proximal end of the braid and the distal end of the helical coil to improve ease of deployment and retrieval of the occlusion implant into and out of the delivery catheter.

In another embodiment, the braided member is formed from a plurality of strands made of a monofilament wire having a closed pitch and braid angle of 35 degrees or less in the collapsed configuration inside the delivery catheter. Such braid may have between 8 and more than 200 strands. The braided member may be configured to have an expanded braid angle between about 25-120 degrees and a diameter between about 0.5 mm to about 50 mm or more.

In another embodiment of the present invention, the occlusion implant includes bioactive coating.

In another embodiment of the present invention, an occlusion device or system for occluding endovascular defect comprises a delivery catheter having a distal end and a proximal end, an elongate occlusion implant extending longitudinally within the delivery catheter and configured to be pushed through and out of the delivery catheter and retrieved back into the distal end of the delivery catheter using a pushing member. The occlusion implant comprises a plurality of regions with at least the first distal region comprised of a non-expandable helical coil and the second elongate region proximal to the first distal region comprised of an expandable braid. The occlusion implant traverses concomitant bends as the delivery catheter when pushed through the delivery catheter to the treatment area.

In another embodiment, the plurality of radial elongate constraining members along the length of the occlusion implant may be comprised of a bioabsorbable material, such that the constraining members help to minimize friction during delivery, but then dissolve to allow full expansion and greater packing volume of the implant post deployment.

In yet another embodiment, the braid portion of the occlusion device or system comprises a tapering configuration formed during fabrication by the braid being woven over a tapered assembly mandrel. Such tapering configuration may taper down from proximal to distal, from distal to proximal, or have any suitable variations of tapering diameters.

In another embodiment of the present invention, an occlusion device or system for occluding endovascular defects comprises a delivery catheter having a distal end and a proximal end, an elongate occlusion implant extending longitudinally within the delivery catheter and configured to be pushed through and out of the delivery catheter and into the delivery catheter using a pushing member. The occlusion implant comprises an elongate expandable braid with a region having plurality of radial elongate constraining members along its length having different expanded diameters. The occlusion implant traverses concomitant bends as the delivery catheter when delivered through the delivery catheter to the treatment location.

In another embodiment, the occlusion implant is made of a braid and includes an elongate constraining member extending along the occlusion implant having a distal end attached to or near the distal end of the braid, and a proximal end attached to near the proximal end of the braid. Such elongate constraining member may have a relatively straight configuration when the occlusion implant is inside of the delivery catheter, and then assume a wavy configuration when the occlusion implant is outside of the delivery catheter.

In another embodiment, an occlusion device or system for occluding endovascular defects comprises a delivery catheter having a distal end and a proximal end, an elongate occlusion implant extending longitudinally within the delivery catheter and configured to be pushed through and out of the delivery catheter and retrieved back into the delivery catheter using a pushing member. The occlusion implant comprises a plurality of expandable braids and helical coils having at least one elongate constraining member along its length. The occlusion implant and constraining member(s) traverse concomitant bends as the delivery catheter when delivered through the delivery catheter to the endovascular defect.

In yet another embodiment, the occlusion implants of the present invention may include components and materials that promote thrombogenicity with at least one elongate constraining member, and may alternatively or in addition, include thrombogenic polymer fibers.

In another embodiment of the present invention, an occlusion device or system for occluding endovascular defects comprises a delivery catheter having a distal end and a proximal end, an elongate occlusion implant extending longitudinally within the delivery catheter and configured to be pushed through and out of the delivery catheter and retrieved back into the distal end of the delivery catheter using a pushing member. The occlusion implant comprises at least two expandable braids: a first distal expandable braid and a second expandable braid, wherein both expanded braids are configured to have a pre-set expanded longitudinal shape when released from the delivery catheter. The occlusion implant traverses concomitant bends as the delivery catheter when delivered through the delivery catheter to the treatment location.

In yet another embodiment, the occlusion implant has at least two braids connected together or one continuous braid with two different longitudinal diminutions that include the following dimensional options: the distal braid is larger than the proximal braid, the distal braid is smaller than the proximal braid, or the distal braid has the same dimension as the proximal braid.

In another embodiment, an occlusion device or system for occluding defects in humans comprises a delivery catheter having a distal end and a proximal end, an elongate occlusion implant extending longitudinally within the delivery catheter and configured to be pushed through and out of the delivery catheter and retrieved back into the distal end of the delivery catheter using a pushing member. The occlusion implant can partially expand having a larger volumetric area when pushed out of delivery catheter. The occlusion implant may have at least one expandable braid and at least one coil. The braid may have a primary outside diameter and a primary braid angle after being manufactured, and the braid may further be reconfigured to have a secondary braid configuration having a secondary outside diameter that has a smaller braid angle than the primary braid angle, and the expandable braid and coil may be attached together.

In yet another embodiment, there is an intermediate external tube member between the proximal end of the expandable braid and the distal end of the coil to connect the braid and the coil. The proximal end of the braid may be positioned inside the intermediate tube member on one end, and the distal end of the coil may be positioned inside the tube on the opposite end. The intermediate external tube may be made of one of the following materials: polymer, metal, metal alloy, rubber, ceramic or any combination thereof.

In another embodiment, the braid and coil may be in contact, or the braid and coil may be spaced apart.

In another embodiment, the secondary braid angle may be smaller than 60 degrees when in the expanded configuration, and preferably around 50 degrees. The braid may be made in one of the following patterns: 1 over-1 under wire, 2 over-2 under wires, 1 over-2 under wires, 2 over-2 under wires, lover-3 under wires; 2 over-3 under wires, 3 over-3 under wires, 1 over-4 under wire, 2 over-4 under wires, 3 over-4 under wires, 4 over-4 under wires and any combination thereof.

In yet another embodiment, an occlusion device or system for occluding defects in humans comprises a delivery catheter having a distal end and a proximal end, an elongate occlusion implant extending longitudinally within the delivery catheter and configured to be pushed through and out of the delivery catheter and retrieved back into the distal end of the delivery catheter using a pushing member. The occlusion implant at least partially expands having a larger volumetric area when pushed out of delivery catheter. The occlusion implant may be made of at least one expandable braid and one coil. The expandable braid(s) may be configured to have a pre-set expanded longitudinal shape when released from the delivery catheter, and the coil(s) may be at least partially extended inside the braid(s), and the braid(s) and coil(s) are connected together on the proximal end of the braid.

In another embodiment, the coil is extended along the entire braid length. The braid and the coil traverse concomitant bends when pushed through and retrieved back into the delivery catheter.

In another embodiment, the proximal end of the braid is not affixed to the coil and can be re-positioned back and forth along the coil as needed while the distal end of the braid and the coil are affixed together.

In yet another embodiment, the occlusion device or system may be comprised of two separate coils: one proximal coil located proximal to the expandable braid, and one inside coil located inside the braid. The inside coil may be attached to the braid on the distal end and on the proximal end, while the proximal coil is attached to the proximal end of the braid. The inside coil and the proximal coil may have several configurations, including but not limited to, straight, not heat pre-shaped, heat pre-shaped, and combinations thereof.

In another embodiment, an occlusion device or system for occluding endovascular defects comprises a delivery catheter having a distal end and a proximal end, an elongate occlusion implant extending longitudinally within the delivery catheter and configured to be pushed through and out of the delivery catheter and retrieved back into the distal end of the delivery catheter using a pushing member. The occlusion implant may at least partially expand having a larger volumetric area when pushed out of delivery catheter. The occlusion implant may include at least one expandable braid having a distal end and a proximal end and at least one constraining member extended longitudinally. The braid may be configured to have a pre-set expanded shape when released from the delivery catheter. The constraining member may be attached to the distal end of the braid and to the proximal end of the braid and may assume a pre-set expanded shape of the braid when pushed outside the delivery catheter. The braid and the constraining member traverse concomitant bends as the delivery catheter when pushed through and retrieved back into the delivery catheter.

In another embodiment, an occlusion device or system for occluding endovascular defects comprises a delivery catheter having a distal end and a proximal end, an elongate occlusion implant extending longitudinally within the delivery catheter and configured to be pushed through and out of the delivery catheter and retrieved back into the distal end of the delivery catheter using a pushing member. The occlusion implant may at least partially expand having a larger volumetric area when pushed out of delivery catheter. The occlusion implant may have at least one expandable braid having a distal end and a proximal end and at least one constraining member extended longitudinally, the constraining member may be configured to have a pre-set expanded shape when released from the delivery catheter. The constraining member may be attached to the distal end of the braid and to the proximal end of the braid. The expandable braid may assume a pre-set expanded shape of the constraining member when pushed outside the delivery catheter, and the braid and constraining member may traverse concomitant bends as the delivery catheter when pushed through and retrieved back into the delivery catheter.

The constraining member and the braid may also both have thermally pre-shaped configurations, and both assume a similar configuration after release from the delivery catheter.

In another embodiment, the occlusion implant comprises a plurality of braids with varied expanded dimensions.

In another embodiment, an occlusion implant comprises at least one outer expandable braid and one inner expandable braid extending longitudinally inside the outer braid. Both braids may be configured to have some or different pre-set expanded shapes when released from the delivery catheter. The inner braid may be attached to the proximal end of the outer braid and have the distal end free floating inside the outer braid. Alternatively, additional coil may be attached to the distal end of the outer braid.

In another embodiment, a method for occluding endovascular defects is provided that includes placing a delivery catheter having an occlusion device or system at the treatment site, wherein the occlusion device or system comprises an occlusion implant and an attached pusher member. Next, the occlusion implant is deployed into the endovascular defect using the pusher member, and then detached inside the endovascular defect. The occlusion device or system traverses concomitant bends as the delivery catheter before deployment.

In another embodiment, the occlusion implant including a expandable braid and/or a helical coil is pre-shaped into a three-dimensional configuration and, when deployed into the treatment area, anchors into surrounding tissue to fill the space and limit blood flow.

In another embodiment, a method for occluding endovascular defects is provided that includes placing a delivery catheter at the treatment site and introducing an occlusion device or system through the delivery catheter to the treatment site. The occlusion device or system comprises an occlusion implant and has an attached detachable pusher member. The occlusion implant comprises at least one expandable braid and one attached helical coil. The occlusion implant is deployed into the endovascular defect using the pusher member, and then detached inside the endovascular defect. The occlusion assembly traverses concomitant bends as the delivery catheter when introduced through the delivery catheter to the endovascular defect.

In another embodiment, a method for occluding endovascular defects comprises deploying the occlusion implant from the delivery catheter, and detaching the occlusion implant, wherein the occlusion implant at least partially expands creating a larger volumetric area than before deployment from the delivery catheter, and wherein the occlusion implant traverses concomitant bends as the delivery catheter while inside the delivery catheter.

In yet another embodiment, occlusion implants of the present invention are configured to resist unacceptable migration from the treatment site following implantation. Initially, device migration is inhibited by anchoring with tissues/vessel at the implantation site, and then by thrombus formation around the occlusion implant.

In another embodiment, an elongated radiopaque component is extended within the expandable braid that comprises one or more micro-coils placed on the core wire and within the braid structure.

In some embodiments, an occlusion implant is configured to cause an acceptable amount of trauma to tissues at the treatment site upon deployment, which can serve to initiate a localized healing to enhance the growth of new patient tissue at the treatment site.

In another embodiment, a method for occluding endovascular defects comprises deploying an occlusion implant from the delivery catheter and detaching the occlusion implant at the treatment area. The occlusion implant at least partially expands, creating a larger volumetric area than before deployment from the delivery catheter. The distal part of the occlusion implant expands upon release from the delivery catheter while the proximal part of the occlusion implant does not expand upon release from the delivery catheter, and the occlusion implant assumes a pre-set configuration upon release from the delivery catheter. The occlusion implant traverses concomitant bends as the delivery catheter before deployment from the delivery catheter.

In another embodiment, a method for occluding endovascular defects comprises deploying the occlusion implant from the delivery catheter and detaching the occlusion implant at the treatment area. The occlusion implant at least partially expands creating a larger volumetric area than before deployment from the delivery catheter, and the distal part of the occlusion implant has the same size before and after delivery from the delivery catheter while the proximal part of the occlusion implant expands upon release from the delivery catheter. The occlusion implant assumes a pre-set configuration upon release from the delivery catheter; and the occlusion implant traverses concomitant bends as the delivery catheter before deployment from the delivery catheter.

In another embodiment, a method for occluding endovascular defects comprises deploying the occlusion implant from the delivery catheter and detaching the occlusion implant at the treatment area. The occlusion implant at least partially expands creating a larger volumetric area than before deployment from the delivery catheter, the distal part of the occlusion implant is not expandable upon release from the delivery catheter, the mid-portion of the occlusion implant expands upon release from the delivery catheter, and the proximal part of the occlusion implant does not expand upon release from the delivery catheter. The occlusion implant assumes a pre-set configuration upon release from the delivery catheter, and the occlusion implant traverses concomitant bends as the delivery catheter before deployment from the delivery catheter.

The occlusion devices or systems of the present invention may be suitable for any one of the following defects: parent vessel occlusion, cerebral and endovascular aneurysms, arterial-venous malformations, embolism, occlusion of blood flow to tumors, Atrial and Ventricular Septal Defects, Patent Ductus Arteriosus and Patent Foramen Ovale.

### BRIEF DESCRIPTION OF THE DRAWINGS

FIG. 1 is a schematic view of an occlusion device or system of the present invention with an occlusion implant inside a delivery catheter, embodied in the form of a expandable braid in a collapsed configuration.

FIG. 2 illustrates the occlusion device or system of FIG. 1 outside the delivery catheter after it has been released.

FIG. 3A is an example of a expandable braid according to the present invention having an open distal end and a tapered proximal section.

FIG. 3B shows a braided angle between two crossing filaments for a braid according to the present invention.

FIG. 4A is a schematic view of an occlusion implant according to another embodiment having a braid in a released straight configuration with radial restraining members.

FIG. 4B is a schematic view of yet another embodiment of the occlusion implant having a braid in a released tapered configuration with radial restraining members.

FIG. 5 illustrates an alternative embodiment of the occlusion implant made of a distal helical coil and a proximal braid in a released non-shaped configuration.

FIG. 6 illustrates an overall view of an occlusion implant of FIG. 1 with pre-set curves deployed from the delivery catheter.

FIG. 7A shows the delivery catheter with the occlusion implant of FIG. 1 inside positioned at the parent vessel occlusion area.

FIG. 7B shows the occlusion implant of FIG. 7A deployed to create parent vessel occlusion.

FIG. 8 shows the occlusion implant of FIG. 7A deployed into the aneurysm.

FIG. 9A shows the braid of the occlusion implant of FIG. 1 prolapsed when retrieved back into the delivery catheter.

FIGS. 9B and 9C show alternatives for preventing prolapse of the braid when retrieved back into delivery catheter.

FIGS. 10A, 10B and 10C show elongated radiopaque components extended within the braid of FIG. 1.

FIG. 11 shows another embodiment of a partially expandable occlusion implant having a distal coil, an intermediate braid and a proximal coil.

FIG. 12 shows another embodiment of a partially expandable occlusion implant having a distal coil and a proximal expandable tapered braid.

FIGS. 13A, 13B and 13C are cross-sectional views of composite Nitinol wires with a platinum core from any of the occlusion implants shown in FIGS. 1, 2, 3A, 4A, 5, 10A, 11 and 12.

FIG. 14 is a schematic view of yet another embodiment of an occlusion implant.

FIGS. 15A, 15B and 15C are cross-sectional views of alternative configurations for the braids of FIGS. 1, 2, 3A, 4A, 4B, 5, 10A, 11 and 12.

FIG. 16 illustrates an alternative method for connecting the braid with the helical coil.

FIG. 17 is a schematic view of yet a further embodiment of an occlusion implant having a variety of braids.

FIGS. 18A, 18B, 18C and 18D show braids that have been reconfigured from the originally manufactured tubular braid.

FIGS. 19A and 19B show an occlusion implant having a braid with the helical coil extended inside the braid.

FIG. 20 shows the occlusion implant of FIG. 1 with the constraining member extended internally.

FIG. 21 illustrates the TED or occlusion implant having an open-ended braid with an attached coil deployed inside a vessel to be closed.

FIG. 22 illustrates an occlusion implant having a braid with both ends closed and an attached coil deployed inside a vessel to be closed.

FIG. 23 illustrates another embodiment of an occlusion implant with a distal coil and a proximal braid.

FIGS. 24A and 24B illustrate the occlusion implant of FIG. 23 deployed inside an aneurysm sac.

FIG. 25 illustrates an MEF device comprising an expandable braid and a constraining member inside the braid.

FIG. 26 illustrates the MEF device of FIG. 25 deployed inside an aneurysm sac.

FIG. 27 illustrates an MEF device deployed inside the aneurysm sac with multiple constraining members attached to the distal and proximal ends of the MEF device.

FIG. 28 illustrates an MEF device deployed inside the aneurysm sac with multiple constraining members attached on one end to the proximal end of the MEF device and freely positioned inside the braid on the other end.

FIG. 29 illustrates an MEF device deployed inside the aneurysm sac with two constraining members attached on one end to the proximal end of the braid and internally attached to the braid on the other end.

FIG. 30 illustrates an MEF device deployed inside the aneurysm sac with an open-ended braid and two constraining members attached on one end to the proximal end of the braid and internally attached to the braid on the other end.

FIG. 31 shows a dual braid occlusion implant device having an outer braid and an inner braid extending longitudinally inside the outer braid.

FIG. 32 shows an occlusion implant device having a dual braid implant with an attached coil.

FIG. 33 shows an embolization plug assembly according to the present invention.

FIG. 34 shows the plug of FIG. 33 deployed at the treatment area.

FIG. 35 shows a preferred structural configuration of the embolization plug in FIG. 33.

### DETAILED DESCRIPTION OF THE INVENTION

FIG. 1 illustrates a schematic view of an occlusion device or system 100 with an occlusion implant 101 inside a delivery catheter 102. The occlusion implant 101 is shown inside the delivery catheter 102 in a collapsed configuration. The occlusion device 100 comprises the occlusion implant 101, the delivery catheter 102, and the pusher member 103. The occlusion implant 101 comprises two elongate regions including a first distal region made of an expandable braid 104 having a distal end 105 and the proximal end 106, and a second elongate region proximal to the first distal region comprised of a non-expandable helical coil 107 having a distal end 108 and a proximal end 109. A distal tip 110 is formed on the distal end 105 of the braid 104 and prevents the very distal section of the braid 104 from fully expanding when deployed from the delivery catheter 102.

The distal tip 110 may be made of one of the following materials: metal, polymer, rubber, adhesive or a combination thereof. One or more radiopaque markers may be positioned along the implant 101 for a better fluoroscopic visibility during deployment or retrieval of the implant 101 inside the delivery catheter 102 including; a radiopaque marker 111 located on the distal end 105 of the braid 104; and a radiopaque marker 112 located on the proximal end 109 of the helical coil 107. Optionally, another radiopaque marker may be located on the proximal end 106 of the braid 104 (not shown) to enhance fluoroscopic visibility of the proximal end 106 of the braid 104 and the distal end 108 of the helical coil 107. Optionally, a radiopaque solder may be used along the braid 104, including the distal end 105 and the proximal end 106, to enhance radiopacity. An elongate constraining member (see below) may enhance the radiopacity of the occlusion implant by virtue of its composition.

The helical coil 107 may be wound from an extension of one or more of the braid strands (not shown), thereby making the braid 104 and coil 107 a continuous mechanical structure and thus eliminating the need for any additional bond connection between the two.

The occlusion implant 101 may include a plurality of regions including braids 104 and helical coils 107 combined in any suitable order from the distal end to the proximal end (not shown).

The proximal end 109 of the helical coil 107 is attached to a pushing member 103 located at least partially within the delivery catheter 102 that functions to deliver the occlusion implant 101 to the treatment location. The pushing member 103 (pusher) is constructed to push the occlusion implant 101 out of, and to retrieve the occlusion implant 101 back into, the distal end 113 of the delivery catheter 102. The pushing member 103 may be made of one of the following materials: wire, tube, wire strand, metal, metal alloy, polymer, polymer knit or any combination thereof. The distal end 114 of the pushing member 103 is attached to a detachment junction 115. The detachment junction 115 is configured for disconnection of the occlusion implant 101 from the pushing member 103 when the occlusion implant 101 is satisfactorily positioned and ready for deployment at the treatment area.

Detachment methods to disconnect the occlusion implant 101 from the pusher 103 may include but are not limited to electrolyte detachment (electrical current); mechanical detachment (movement, screw or pressure); thermal detachment (localized delivery of heat); and radiation detachment (electromagnetic radiation). The detachment junction 115 may be attached to the occlusion implant 101 directly or by using an intermediate member such as polymer or fiber material (not shown).

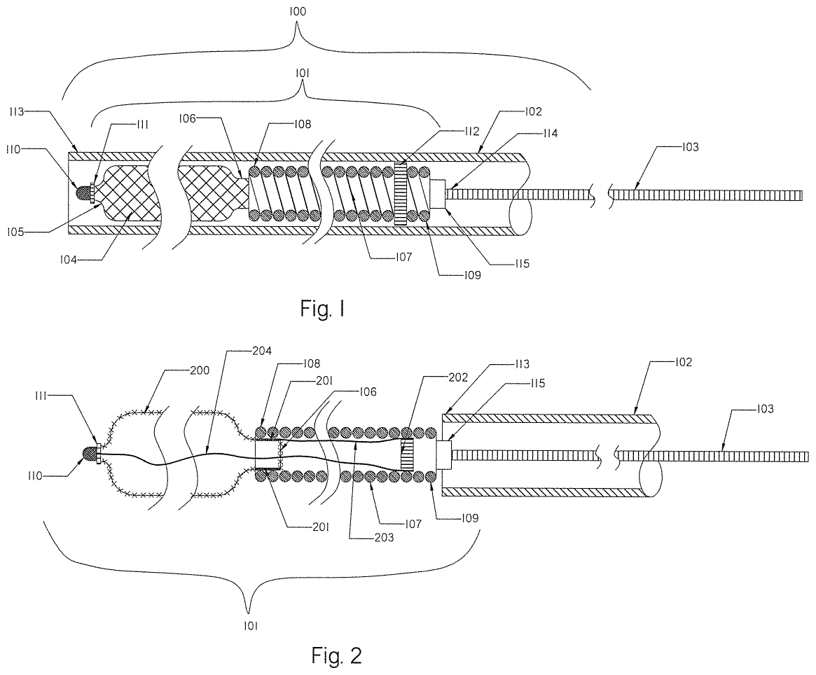

Alternatively, the detachment junction 115 may be positioned anywhere along the length of the occlusion implant 101 (not shown). The distal end 108 of the helical coil 107 is attached to the proximal end 106 of the braid as shown in detail in FIG. 2.

The delivery catheter 102 having a distal end 113 provides a shield and serves as a delivery vehicle to deliver the occlusion implant 101 to the treatment location. The delivery catheter 102 may have an inner diameter between 0.015 inches and about 0.100 inches and its inside layer should preferably be made of a low friction polymer material to ease the delivery of the occlusion implant 101 to the treatment location. Polymer materials having a low friction coefficient may include but are not limited to Teflon, Polyamide, Low Density Polyethylene, Polytetrafluoroethylene (PTFE), Polyoxymethylene (Delrin).

When the occlusion implant 101 is in a compressed configuration as shown in FIG. 1 inside the delivery catheter 102, it traverses concomitant bends in the same manner as the delivery catheter 102 during positioning at the treatment location.

FIG. 2 is an internal view of the inside of the occlusion device or system 100 with the occlusion implant 101 deployed outside of the distal end 113 of the delivery catheter 102. The distal braid 104 as shown in FIG. 1 now has an expanded configuration 200. The expanded braid 200 may have a diameter that is at least 1.3 times larger than the diameter of the second region helical coil 107 when the occlusion implant is released from the delivery catheter 102.

The proximal end 106 of the expanded braid 200 is connected to the distal end 108 of the helical coil 107 via a connecting feature 201. The proximal end 106 of the braid 200 may be positioned either inside of the distal end 108 of the helical coil 107 or overlap the distal end 108 of the helical coil 107 (not shown). The connection feature 201 between both sections may be formed by one or more of the following methods; bonding, fusing, welding, soldering, gluing, other mechanical means or any combination of all.

The expandable braid 104 of the occlusion implant 101 has a greater length when at its collapsed configuration inside the delivery catheter 102 as shown on FIG. 1 than when it is expanded as the deployed braid 200 outside the distal end 113 of the delivery catheter 102. In addition, a radiopaque marker 202 may be placed inside the proximal end 109 of the helical coil 107 and/or inside the proximal end 106 of the braid 104 (not shown). As shown in FIG. 1 and FIG. 2 radiopaque marker 202 may be positioned inside the proximal end 109 of the helical coil 107, on the outside surface of occlusion implant 101 at 112 and 111 locations or on both locations. Alternatively, radiopaque soldering may be used to enhance radiopacity in any location along the occlusion implant 101 including braid 104 and helical coil 107.

At least one elongate constraining member 203 that prevents the helical coil 107 from stretching is extended through the helical coil 107 and it is attached to or near the distal end 108 of the helical coil 107 and to or near the proximal end 109 of the helical coil 107. Alternatively, or in addition, at least one elongate constraining member 204 may be extended through the occlusion implant 101 to prevent the whole implant from stretching and from damage. The constraining member 204 may be attached at one end to or near the distal tip 110 of the braid 104, and at other end proximally to or near the proximal end 109 of the helical coil 107. The elongate constraining members 203 and 204 may be made of a single wire, multiple wires, strands, coils, tubes, polymer rod, knit, woven, braid and have several configurations including but not limited to: straight, bent, coiled, helical, sinusoidal, wave or any combination thereof. Such elongate constraining members may be made of metal, metal alloy, polymer or a combination of the above.

The elongate constraining members 203 and 204 may have variable stiffness along their length, such as stiffer distally and more flexible proximally, stiffer proximally and more flexible distally, or a stiffness that constantly changes along its length. Alternatively, the elongate constraining members may comprise of a plurality of members made of wire, strands, coils, tubes, polymer rod, braid attached together, optionally including radiopaque members.

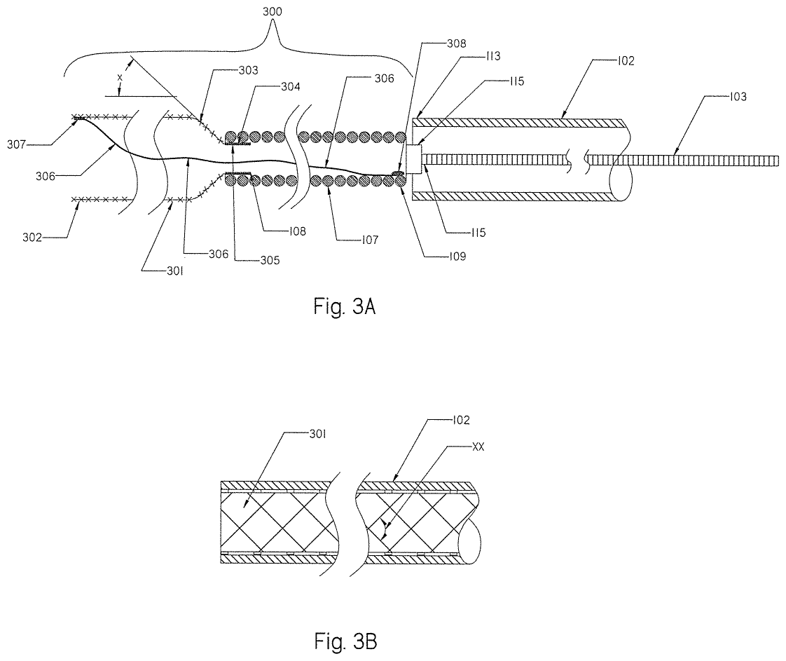

FIG. 3A illustrates an alternative configuration of the occlusion implant 300 that comprises a distal braid 301 having an open distal end 302 and a tapered proximal end 303 affixed proximally to the distal end 108 of the helical coil 107 at location 304. Tapered proximal section 303 of the braid 301 is preferably provided at any angle between 15-45 degrees as shown by the angle X. Such tapered portion 303 facilitates ease of deploying and retrieving the proximal end 303 of the braid 301 into or outside the distal end 113 of the delivery catheter 102, lowering tension forces that are created between the larger size braid 301 when it is pulled inside a smaller size delivery catheter 102 in an expanded configuration. Attachment location 304 is configured by overlapping the distal-most end 108 of the helical coil 107 over the proximal-most end 305 of the braid 301 and attaching both together using similar attachment methods as described for the attachment 201 in FIG. 2.

The open-ended braid 301 will enhance engagement of its distal end 302 into the tissue within the treatment area and serve as a distal anchor of the implant 300. There is no safety issue of perforating the treatment area with an open braid 302 because the opening or terminating strands of the occlusion implant 300 are made of a very fine wire.

The expandable braids of the present invention may be made of a plurality of wire strands having a thickness that is between about 0.0005 inches and about 0.010 and the same dimensions or different dimensions braided into the desirable shape. The expandable braids of the present invention may be constructed of wire strands made of the following materials: metals, alloys, polymers, a shape memory material (e.g., Nitinol), cobalt-chromium alloys, Platinum, Platinum-Iridium alloys, polymers (e.g., Nylon, Polyester, etc.) or combinations of any. The expandable braid may be formed from a plurality of wires having multiple wire strands of the same dimensions or different dimensions braided into the desirable shape using circular wire, oval wire, fiat wire and any other suitable wire configuration. The helical coil may be formed from a single wire or a plurality of wires having the same dimensions or different dimensions using circular wire, oval wire, flat wire and any other suitable wire configuration.

The braids 104, 301 may be formed from a plurality of strands made of a monofilament wire having a closed pitch and braid angle of 35 degrees or less in the collapsed configuration when inside the delivery catheter. Braid angle XX as shown in FIG. 3B is the angle between two crossing filaments of the braid. The braid 104, 301 may be configured to have an expanded braid angle between about 35-90 degrees (not shown).

The overall radial diameters of the braid 301 of the occlusion implant 300 in the expanded position as shown in FIG. 3A may be between about 0.5 mm to about 20 mm or more. Such tubular braid may have between 8 and 200 or more strands, and preferably 24 to 72 strands.

The helical coils of the present invention may be wound from one or more wires made from one of the following materials: metals, alloys, polymers, shape memory materials (e.g., Nitinol), cobalt-chromium alloys, Platinum, Platinum-Iridium alloys, polymers (e.g., Nylon, Polyester, etc.) or combinations of any.

The helical coil may be prepared by wrapping a suitable wire about a cylindrical or conical mandrel. Any loose end of a helical wire coil may be placed axially through the core of the helix and bound to another part or coil using, e.g., by heat, adhesives, and/or mechanical means. Alternatively, or in addition, a thrombogenic element (e.g., particles, radial filaments, polymer fibers etc.) may be attached to portions of the coil 107 by tying/adhering them to the coil 107 (not shown). The elongate constraining member 306 is attached to or adjacent the distal end 302 of the open braid 301 at the attachment area 307 and to (or adjacent) the proximal end 109 of the helical coil 107 at the attachment point 308 using conventional attachment methods, including but not limited to bonding, welding, and heat fusing.

Additional thrombogenic elements (e.g., particles, radial filaments, polymer fibers etc.) may be attached to at least a portion of the elongate constraining member 306 using any suitable binding technique; e.g., by tying or otherwise adhering them to the elongate constraining member 306 (not shown).

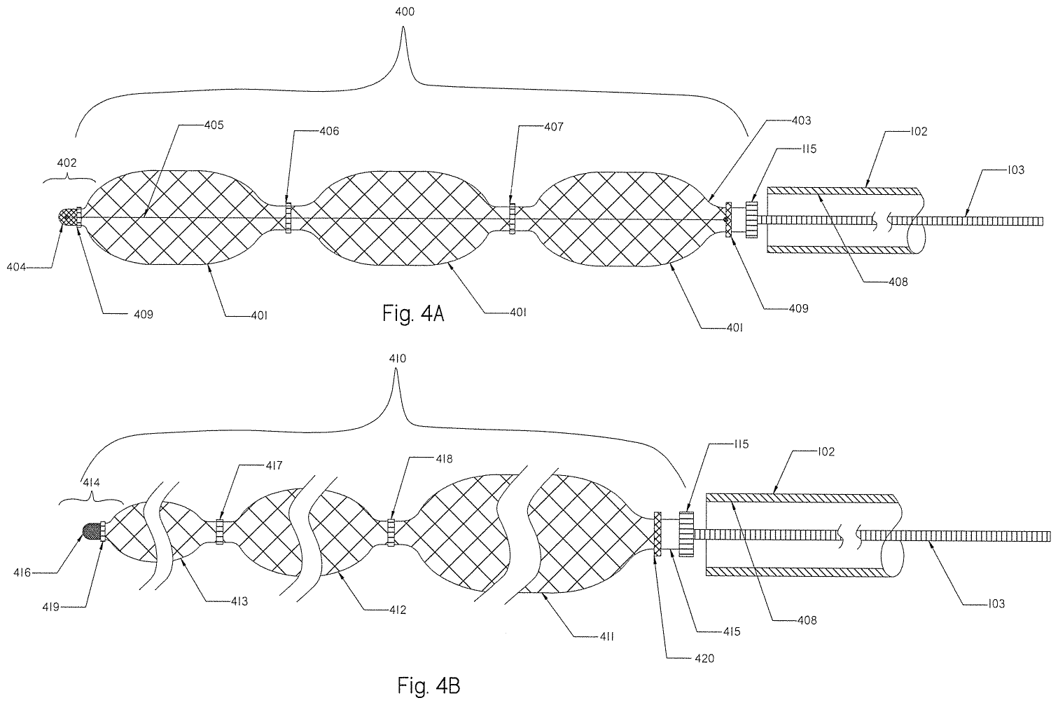

FIG. 4A shows an alternative version of the occlusion implant 400 comprising a braid 401 having a distal end 402 and proximal end 403. The distal tip 404 is attached to the distal end 402 of the braid 401 to prevent the very distal section of the braid 401 from fully expanding when deployed from the delivery catheter 102. The tip 404 may be made from the same material as the tip 110 described in FIG. 1. One or more longitudinal restraining/constraining members 405 may be located inside the occlusion implant 400. One or more radial constraining members 406, 407 are positioned along the braid 401 to restrain the outside dimension of the braid 401, thereby facilitating and easing the deployment and retrieval of the braid 400 into and out from the delivery catheter 102. A smaller radial dimension of the braid 401 at radially constraining areas 406 and 407 will also reduce tension forces of the braid 401 between the inner wall 408 of the delivery catheter. 102 and the outer surface of the braid 401. Radial constraining members 406 and 407 may also serve as radiopaque markers for a better visualization of the occlusion implant 400 during deployment and retrieval. Additional radiopaque markers 409 may be positioned on the distal and proximal ends of the implant 400 to provide complete visibility of the implant 400 along its length. Such a braid 400 may include helical coils attached either on the distal end, the proximal end, or on both ends (not shown).

The delivery of the occlusion implant 400 to the treatment area and outside of the delivery catheter 102 becomes more difficult when friction between the outer surface of the braid 401 and the inner wall 408 of the delivery catheter 102 is high. The longer the occlusion implant 400 is, and the bigger the outer diameter of the braid 401 in the expanded configuration, the more challenging the delivery and retrieval of the occlusion implant 400 would be. Both these attributes (occlusion implant length and expanded braid size) play a very important role in clinical applications because a greater implant volumetric size will facilitate better occlusion implant engagement structure/edifice, and the larger surface area for promotion of blood clotting.

Use of a surface coating may be helpful to reduce friction between the braid 401 and the inner wall 408 of the delivery catheter 102. All or part of the outer surface of the occlusion implant 400 may be coated with Parylene (poly paraxylylene) or any other suitable polymers to reduce the friction coefficient when the occlusion implant 400 is deployed outside of the delivery catheter 102 or retrieved inside of the delivery catheter 102.

FIG. 4B shows an alternative version of a tapered occlusion implant 410 that comprises a plurality of braids, including but not limited to: a proximal braid 411, an intermediate braid 412, and a distal braid 413. The occlusion implant 410 has a distal end 414 and a proximal end 415. The distal braid 413 is smaller (e.g., smaller diameter) than the intermediate braid 412, which is smaller (e.g., smaller diameter) than the proximal braid 411, in the expanded configuration. Different dimensions between these three braid regions may be achieved by appropriate sizing of the assembly mandrel, pre-shaping of the braid sections, or both. Any suitable combination of sizing for the braid sections may be considered when needed, including a larger braid on the distal end, a larger braid in the middle or a smaller braid on the proximal end, depending on clinical needs (not shown). Such positioning of the braids along, or in combination with, coils may provide more effective filling of the aneurysm. Some aneurysm anatomies, for example, may have a sac narrowing away from the neck, and in such a case, a distal coil may provide a better option for filling such space. In some other cases, the aneurysm may have a spherical or orbicular shape, and in such a case, a distal braid may fill such space more effectively. In any case, a proximal coil will provide a finishing aneurysm filler and seal, thus, preventing blood penetration inside the aneurysm. Such diversified braid sections and braid sizing may further improve and facilitate the deployment and retrieval of the implant 410 from and into the delivery catheter 102. The distal tip 416 is attached to the distal end 414 of the distal braid 413 to prevent the very distal section 414 of the braid 410 from fully expanding when deployed from the delivery catheter 102. The tip 416 may be made from the same material as the tip 110 described in FIG. 1.

One or more radial constraining members 417, 418 are positioned along the braid 410 to restrain the outside dimensions of the braid 410, thereby facilitating and easing the deployment and retrieval of the braid 410 into and out from the delivery catheter 102. A smaller radial dimension of the braid 410 at radially constraining areas 417 and 418 will also reduce braid tension forces between the inner wall 408 of the delivery catheter 102 and the outer surface of each braid segments 411, 412 and 413. Radial constraining members 417 and 418 may also serve as radiopaque markers for a better visualization of the occlusion implant 410 during deployment and retrieval. A distal radiopaque marker 419 and a proximal radiopaque marker 420 provide complete visibility of the implant 410 along its length. Such a braid 410 may include helical coils attached either on the distal end, the proximal end, or on both ends (not shown).

The construing members 406 and 407 in FIG. 4A and constraining members 417 and 418 in FIG. 4B located along the length of the occlusion implant may be comprised of a bioabsorbable material, such that the constraining members help to minimize friction during delivery, but then dissolve to allow full expansion and greater packing volume of the implant post deployment.

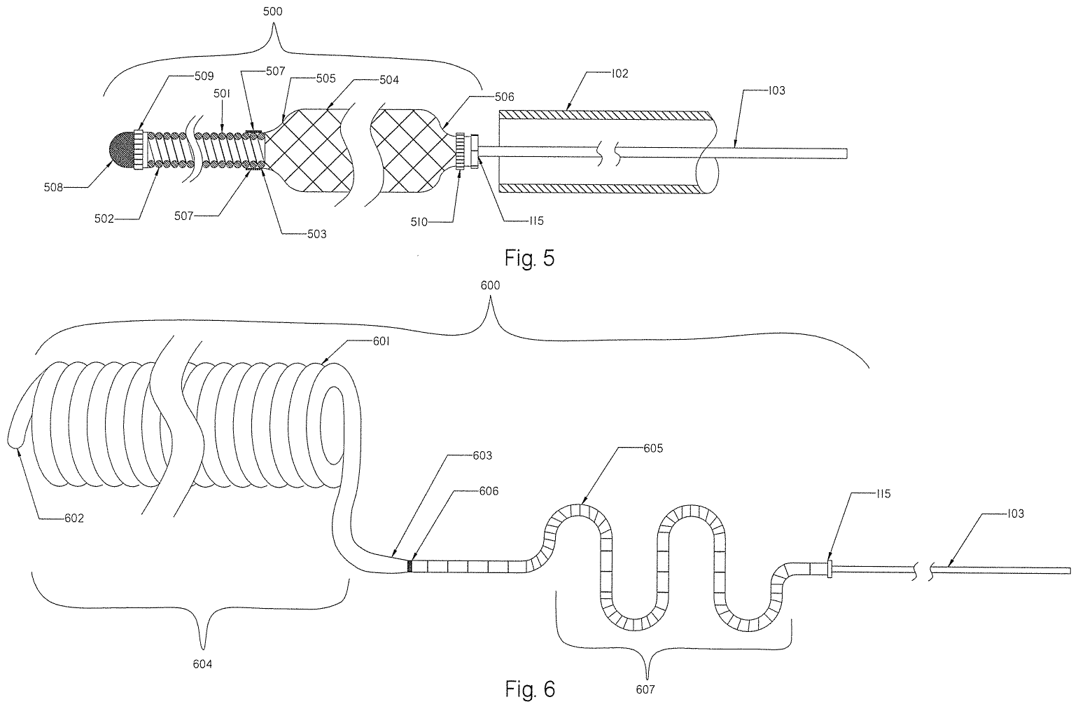

FIG. 5 shows an alternative version of the elongated occlusion implant 500 that comprises a distal helical coil 501 having a distal end 502 and a proximal end 503, followed by a braid 504 having a distal end 505 and a proximal end 506. The distal end 505 of the braid 504 overlaps the proximal end 503 of the helical coil 501 and both members are attached together at location 507. The distal tip 508 is attached to the distal end 502 of the helical coil 501. The tip 508 may be made of similar material and attached with similar methods as the tip 110 in FIG. 1. A radiopaque marker 509 may also be attached to the distal end 502 of the helical coil 501. Alternatively, or in addition, other radiopaque markers may be attached along the occlusion implant 500 including, but not limited to, a proximal marker 510 attached to the proximal end 506 of the braid 504. At least one constraining member may be attached internally within the helical coil 501 alone, or to the helical coil 501 and braid 504 if necessary, to prevent the implant structure from stretching (not shown). Alternatively, the occlusion implant 500 may comprise a plurality of consecutive helical coils and braids attached in any desirable order (not shown).

Occlusion implants of the present invention may be coated internally and/or externally with bioactive agents consisting of a growth factor, a protein, a proteoglycan, a glycosaminoglycan, a physiologically compatible mineral, an antibiotic, a chemotherapeutic agent, a pharmaceutical, an enzyme, a hormone, and genetic material. Alternatively, occlusion implants may include bioactive coatings immobilized on a surface of the occlusion implants. The coating material may include a biotropic ECM (extracellular matrix), with a network of self-assembled collagen fibrils and at least one bioactive agent retained in the ECM material. The coating material may coat the entire surface of the occlusion implant, or any portion thereof, and may comprise one or more individually formed ECM material layers.

Occlusion implants may include material that promotes thrombogenicity including, but not limited to, yarns, fibers, and/or resins, e.g., monofilament yarns, polyester, and the like, as well as other plastic, resin, polymer, woven, fabric surgical materials, shape-memory plastics, and combinations of such materials.

FIG. 6 shows an occlusion implant 600 having a plurality of pre-set shapes. The expanded braided member 601 of the occlusion implant 600 has a distal end 602 and a proximal end 603. In addition, or alternatively, the braided member 601 may have a pre-set secondary or tertiary shape (not shown). The attached helical coil 605 has a distal end 606 that is attached to the proximal end 603 of the member 601. The helical coil 605 may also have a pre-set sinusoidal shape 607 or any other desirable shape that can serve as a volumetric filler. A whole elongate length of the occlusion implant 600, including the braided member 601 and helical coil 605, may also be configured along its length to have a variety of a pre-set curves or shapes including sinusoidal shape, curved shape, and spherical shape, among others.

The occlusion implants of the present invention may be introduced into a patient via a catheter inserted into the treatment area to treat parent vessel occlusion or to occlude an aneurysm. At either treatment site, the occlusion implant may be pushed distally out of the catheter and delivered into the parent occlusion site or aneurysm. After being deployed from the catheter, the braided portion of the implant will self-expand into the expanded configuration and assume a pre-set configuration as described above. The deployment of the occlusion implant is always observed under fluoroscopy, and in case the occlusion implant deployment is not satisfactory, the occlusion implant may also be removed or withdrawn (collapsed back into the delivery catheter) and removed outside the body if necessary.

Any of the occlusion implants described in the present invention may be inserted into endovascular and non-endovascular defects, including arteries and veins for parent vessel occlusion or into an aneurysm in order to occlude the aneurysm. The occlusion implant having an expandable braid may have numerous advantages compared to existing therapies such as coils/stents/plugs for shutting the parent vessel or filling aneurysms. The expandable braid would provide many times greater volumetric filing, that may quickly and constantly occlude the artery or divert blood flow from the aneurysm entry, thus reducing the number of coils required per closing of the parent artery or filling of the aneurysm. It may also reduce the risk of aneurysm recanalization, which may allow a patient to avoid taking anti-platelet medications or blood thinners.

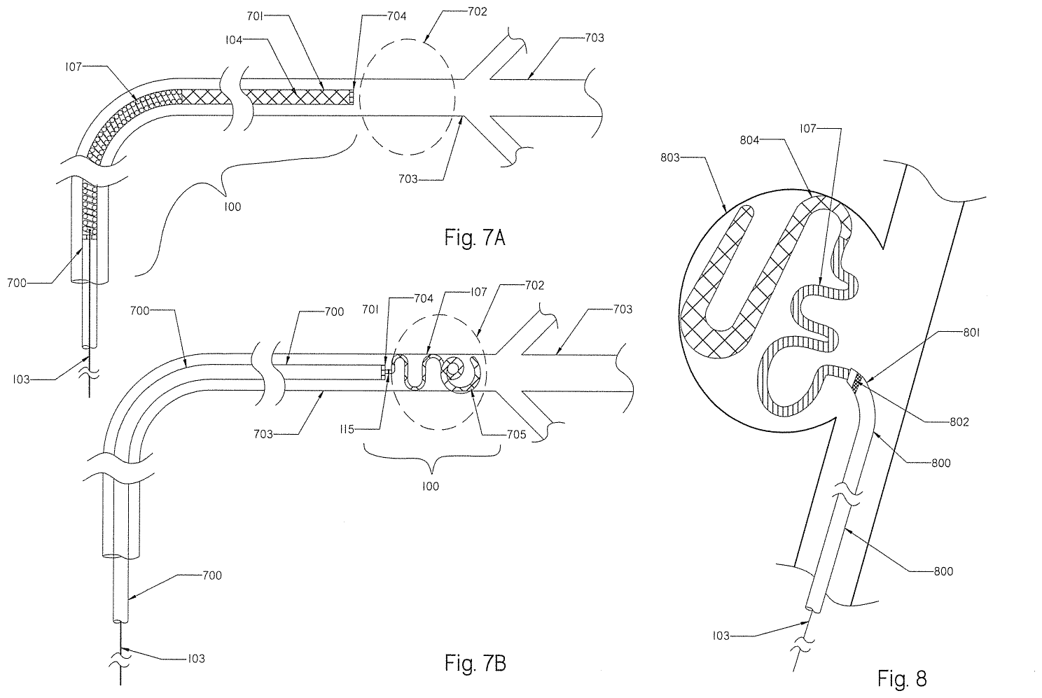

FIG. 7A shows a delivery catheter 700 having a distal end 701 positioned at the treatment location 702 of a parent vessel 703. A radiopaque marker 704 is located on the distal end 701 of the delivery catheter 700. The occlusion implant 100 of FIG. 1 is located distally inside the delivery catheter 700. When the delivery catheter 700 traverses bends and anatomical curves to access the treatment location 702, the occlusion implant 100 traverses the same concomitant bends as the delivery catheter 700 during its delivery to the treatment location 702. Also, during the movement of the occlusion implant 100 within the delivery catheter 700 in either distal/proximal or proximal/distal directions, the occlusion implant 100 traverses the same concomitant bends as the delivery catheter 700 during such movements. When the distal end 701 of the delivery catheter 700 is satisfactorily positioned at the treatment location 703, the occlusion device/system 100 is deployed by moving the pushing member 103 and the implant 100 distally into the treatment area 702 of the parent vessel 703 as shown in FIG. 7B. The detachment 115 of the pusher member 103 and the proximal coil 107 is inside the treatment area 702, and is ready for detachment. The pusher member 103 traverses concomitant bends as the delivery catheter 700 during its delivery to the treatment location 702. Upon deployment of the occlusion implant 100 into the treatment area 702, the distal braid 104 expands into an expanded configuration 705, assuming a pre-set shape and anchoring into the wall of the treatment area 702. The helical coil 107 further fills the space of the treatment area 702.

The occlusion implant 100 may also be withdrawn and collapsed back into the delivery catheter 700 in case the deployment of the implant 100 into the treatment area 702 is not satisfactory. The placement of the occlusion implant 100 inside the treatment area 702 may be repeated multiple times until the correct position is achieved. When the braid 104 expands inside the treatment area and reaches an expanded configuration 705 and pre-shaped contour, it begins to occupy a greater space within the treatment area 702, providing engagement structure for the helical coil 107 to further fill the treatment space and the promotion of blood clotting. Once the position of the occlusion implant 100 is satisfactory within the treatment location 702, the occlusion implant is disconnected (detached) from the proximal end 109 of the helical coil using the detachment junction 115 as shown in FIG. 2.

FIG. 8 shows a delivery catheter 800 having a distal end 801 with the radiopaque marker 802 positioned at the aneurysm sac location 803. When the distal end 801 of the delivery catheter 800 is positioned satisfactorily at the aneurysm sac 803, the occlusion implant 101 having a distal braid 104 and the proximal helical coil 107 as shown in FIG. 1 is deployed into the aneurysm sac 803 using the pushing member 103. Once the occlusion implant 101 is deployed, the distal braid goes into expanded configuration 804 and assumes a pre-set shape while the helical coil 107 follows at its pre-set configuration and fills the aneurysm sac 803. The implant 101 may also be removed or withdrawn and collapsed back into the distal end 801 of the delivery catheter 800 if the position of the occlusion implant 101 within the aneurysm sac 803 is not satisfactory. The expanded braid 804 begins to occupy a greater space within the aneurysm sac 803, providing engagement structure for the helical coil 107 to further fill the aneurysm sac 803 and the promotion of blood clotting. When the delivery catheter 800 traverses bends and anatomical curves to access the aneurysm, the occlusion implant 101 in its collapsed configuration traverses concomitant bends as the delivery catheter 800.

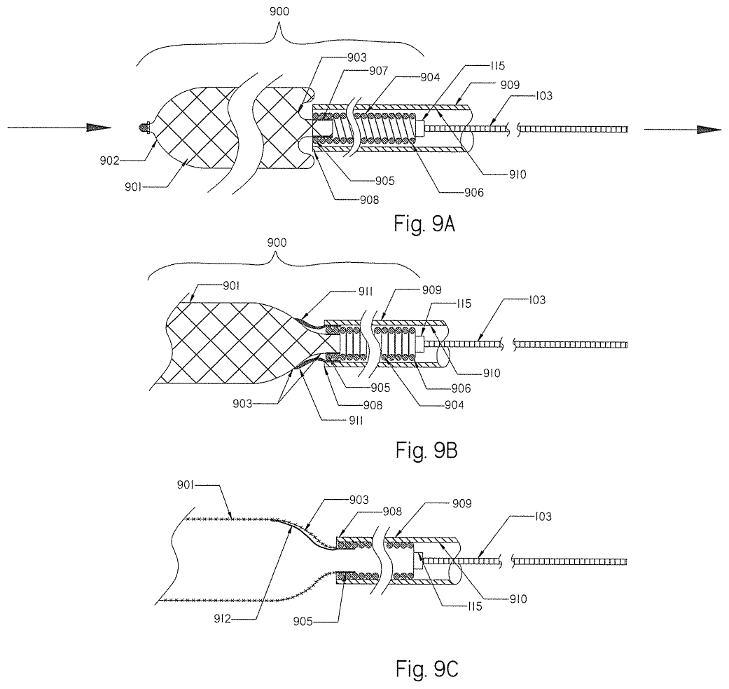

FIG. 9A shows an occlusion implant 900 that comprises a braid 901 having a distal end 902 and a proximal end 903. A helical coil 904 has a distal end 905 and a proximal end 906. The proximal end 903 of the braid 901 is attached to the distal end 905 of helical coil 904 at the attachment connection 907. When the occlusion implant 900 is retracted (as shown by the moving direction arrows in FIG. 9A) into the distal end 908 of a delivery catheter 909, the proximal end 903 of the braid 901 may produce a serious frictional interface between the proximal end 903 of the occlusion implant 901 and inner lumen/surface 910 of the delivery catheter 909, and often cause prolapse of the proximal braid 903 over the distal end 908 of the delivery catheter 909. Consequently, the occlusion implant 900 may become damaged, broken or otherwise not functional.

FIG. 9B shows an additional option to those shown in FIG. 3A and FIG. 3B to reduce friction and improve the movement of the occlusion implant 900 into the distal end 908 of the delivery catheter 909. A small wall thickness shrink tubing 911 may be placed over the proximal portion 903 of the brad 901 and partially over the distal end 905 of the helical coil 904. Such shrink tubing 911 or any other similar polymer sleeve will further strengthen the proximal portion 903 of the braid 901, thereby reducing interface friction between the proximal end 903 of the braid 901 and the inner lumen/wall 910 of the distal end 908 of the delivery catheter 909.

FIG. 9C shows an alternative or additional elongate constraining member 912 that may be attached to the proximal end 903 of the braid 901 and the distal end 905 of the helical coil 904. The elongate constraining member 912 may be made of metal wire, polymer, braid or a combination of all. The constraining member 912 will stiffen the proximal end 903 of the braid 901, ease movement between parts, and consequently improve movement. Other means to improve the retrieval of the braid 901 into the distal end 908 of the delivery catheter 909 may include, but are not limited to: (i) friction reduction surface coating of the proximal end 903 of the braid 901, (ii) pre-shaping the proximal end 903 of the braid 901 at an angle that is less than 45 degrees (as described in FIG. 3A), (iii) braid angulation as described in FIG. 3B, (iv) friction reduction coating of the inner lumen 910 of the delivery catheter 909, and (v) other suitable methods.

The configuration of the braid 901 may be formed during fabrication by the braid being woven over a tapered assembly mandrel. Such tapering configuration may taper down from proximal to distal, from distal to proximal, or in any suitable combination of tapering diameters.

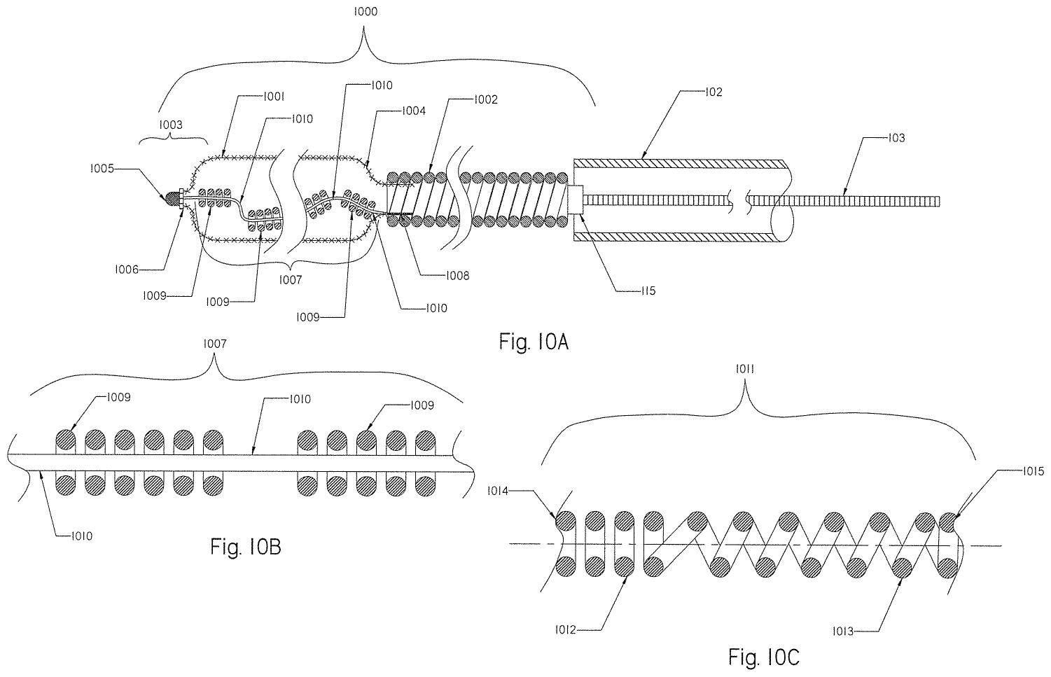

FIG. 10A is a cross-sectional view of an occlusion implant 1000 having a braid 1001 attached to a helical coil 1002. The braid 1001 has a distal end 1003 and a proximal end 1004. The distal tip 1005 is attached to the distal end 1003 of the braid 1001. A radiopaque marker 1006 is also attached to the distal end 1003 of the braid 1001. A radiopaque component 1007 (as shown in FIG. 10B) is attached to the distal end 1003 of the braid 1001 and to the proximal end 1004 of the braid 1001.

The radiopaque component 1007 comprises at least one or more radiopaque helical micro-coils 1009 positioned over a core wire 1010. The micro-coils 1009 may be made of any suitable radiopaque material including but not limited to platinum or gold. The core member 1010 may be made of polymer, metal or metal alloy, including but not limited to suture, SST or Nitinol as a single or multi member unit including wire strands. One or more micro-coils 1009 may be freely placed over the core wire 1010, so it can move along the core wire 1010. The micro-coils 1009 may also be attached to the core wire 1010 using any suitable means, such as glue, crimp, soldering or other means (not shown). In the collapsed position when the braid 1001 is inside the delivery catheter 1.02, the radiopaque component 1007 assumes a relatively straight configuration (not shown). When the braid 1001 is in the expanded configuration, the radiopaque component 1007 assumes a wavy configuration.

FIG. 10C shows an alternative radiopaque component 1011 which is made of a stretchable helical coil having at least one or more closed coil sections 1012 and one or more open coil sections 1013. The distal end 1014 may be attached to the distal end 1003 of the braid 1001, while the proximal end 1015 may be attached to the attachment point 1008 (not shown). When the braid 1001 is inside the delivery catheter 102, the open coil section 1013 is stretched between the distal end 1003 of the braid 1001 and the proximal end 1004 of the braid 1001, and when the braid 1001 is in the expanded configuration, the open coil section compresses and may assume a wavy configuration (not shown). Also, the radiopaque component 1011 may be made of a single stretchable helical coil that is on one end attached to the distal end 1003 of the braid 1001, and on the other end attached to the attachment point 1008 (not shown). Such a radiopaque stretchable coil will be in a stretched position when the braid 1001 is collapsed inside the delivery catheter 102 and is in a compressed position when the braid 1001 is expanded outside the delivery catheter 102 (not shown).

FIG. 11 shows an alternative version of a partially expandable occlusion implant 1100 that includes a distal helical coil 1101 having a distal tip 1106, a braid 1102, and a proximal helical coil 1103. The proximal helical coil 1103 is connected to the braid 1102 at the connection area 1105. The distal helical coil 1101 is connected to the braid 1102 at the connection area 1104. The distal helical coil 1101 may be larger, smaller or have the same outside dimension as the proximal coil 1103. Distal coil 1101 and proximal coil 1102 may have the same length or different lengths, and can be made the same or different wire shape and material.

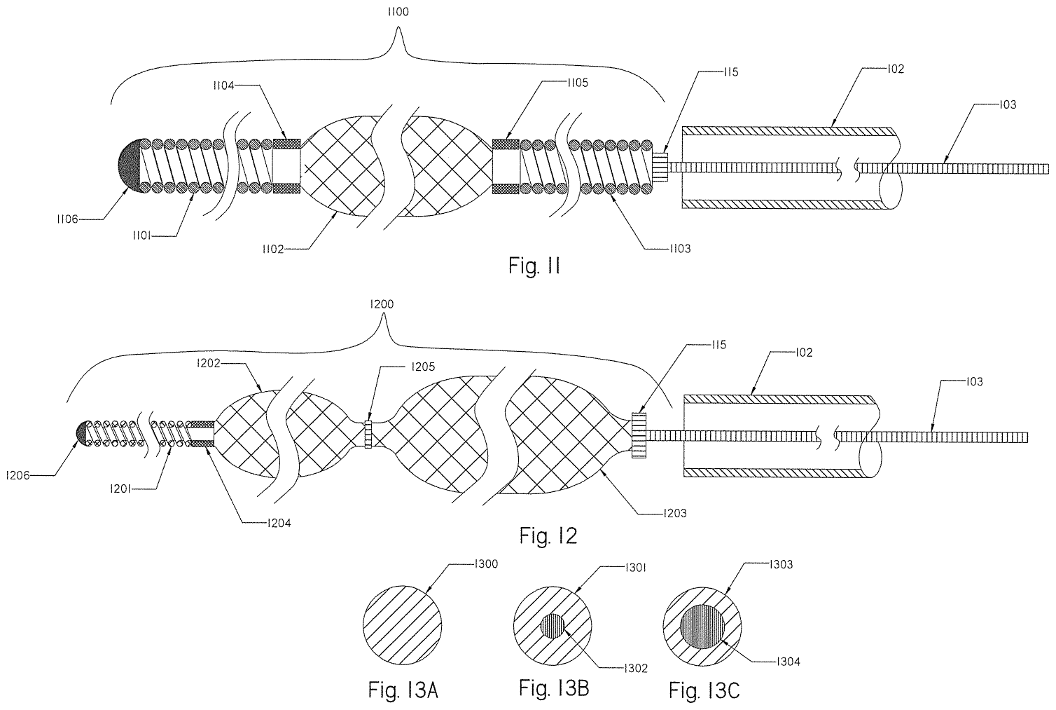

FIG. 12 shows another alternative version of a partially expandable occlusion implant 1200 that includes a distal coil 1201, a first proximal braid 1202 and a second proximal braid 1203. The distal helical coil 1201 has a distal tip 1206. The first proximal braid 1202 is connected to the distal helical coil 1201 at the connection area 1204. The first braid 1202 is connected with the second braid 1203 at the connection area 1205. In another embodiment, the first proximal braid 1202 and the second proximal braid 1203 may be made of one member having the first proximal braid 1202 smaller (e.g., smaller diameter) than the second proximal braid 1203. Such a configuration of the braid with the more distal braid (1202) smaller than the more proximal braid (1203) may greatly improve ease of delivery, deployment and retrieval of the occlusion implant 1200 to and from a treatment location.

To further increase or improve radiopacity of the braids of the present invention, the Nitinol wires used to make the braids may be made as composite wires with 10-30% platinum. FIG. 13A shows a cross section of a conventional Nitinol wire 1300 without any radiopaque core material. FIG. 13B shows a Nitinol tube 1301 filed with platinum core 1302 that represents approximately 10% of the overall cross section of the composite wire. FIG. 13C shows a Nitinol wire 1303 with approximately 30% of the platinum core 1304. Such composite Nitinol/Platinum wires including 10-30% Platinum are made by Fort Wayne Corporation, Ind.

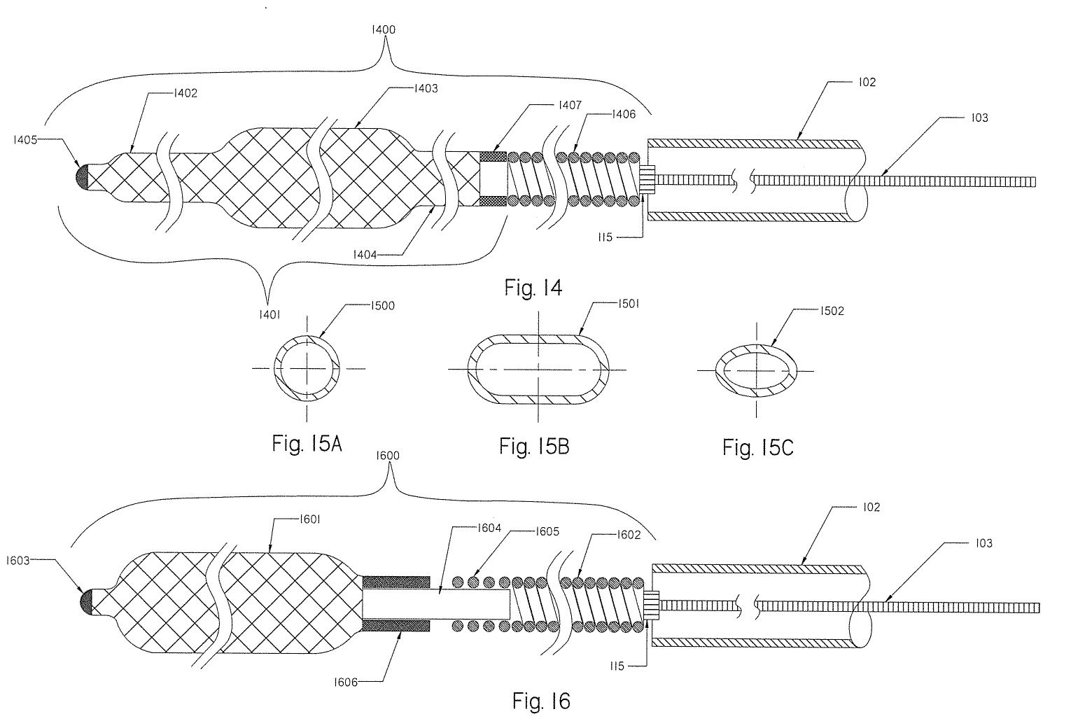

FIG. 14 illustrates an alternative configuration of a braid 1401 that is suitable for improving and easing the deployment and retrieval of an occlusion implant 1400 into the delivery catheter 102. The occlusion implant 1400 comprises a braid 1401 having a distal section 1402, a mid-section 1403, a proximal section 1404, and a distal tip 1405. The distal section 1402 is smaller (e.g., has a smaller diameter) than the mid-section 1403 in the expanded configuration. The proximal section 1404 is also smaller (e.g., has a smaller diameter) than the mid-section 1403 in the expanded configuration. In one embodiment, the distal section 1402 and the proximal section 1404 may have the same continuous outside dimensions. In another embodiment, the distal section 1401 may be tapered down distally towards the tip 1405 (not shown), while the proximal section 1404 may be tapered down proximally towards the helical coil 1406 (not shown). In yet another embodiment, the distal section 1402 may have a continuous outside dimension, while the proximal section 1404 is tapered down proximally towards the helical coil 1406 (not shown). There is a connection area 1407 that connects the proximal end of the braid 1401 and the distal end of the helical coil 1406. Using a tapered distal section 1402 of the braid 1401 will ease the deployment of the occlusion implant 1401. Retrieval of the occlusion implant 1401 into a delivery catheter is usually easier than deployment because it is pulled back into the delivery catheter 102, and in such cases, significant pulling forces may be used without risk of damaging the occlusion implant 1400.

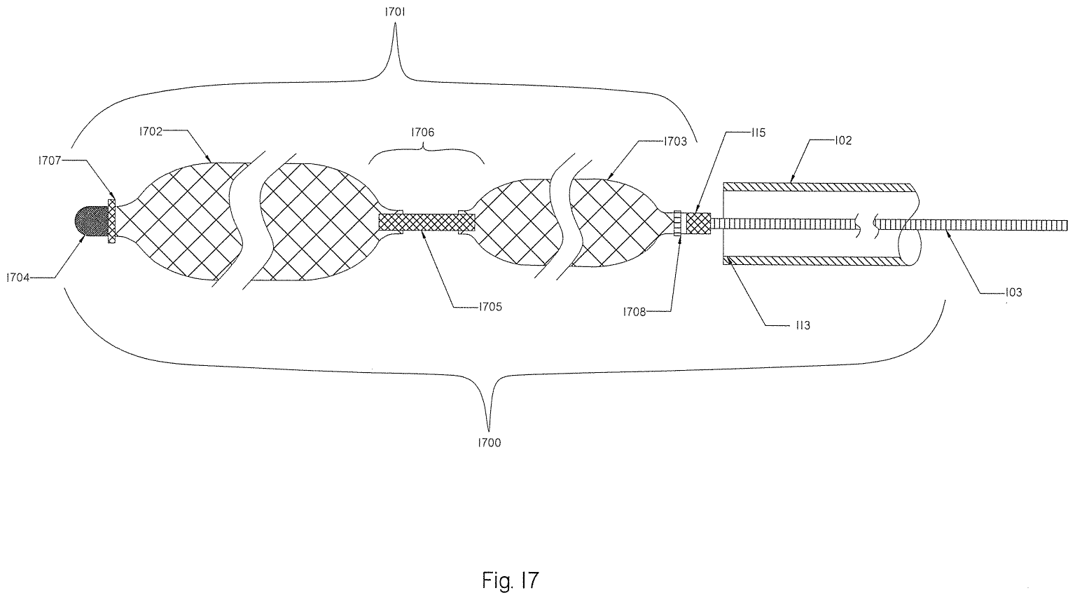

FIG. 16 illustrates an alternative method for connecting the braid to the helical coil. It is important to maintain the smallest outside diameter for the connection area as possible to ease the movement of the implant within the catheter 102. The occlusion implant 1600 comprises a braid 1601 (having a distal tip 1603) and a helical coil 1602 that are connected using an intermediate internal member 1604. Such intermediate internal member 1604 may have any suitable shape or configuration, may be made of metal or plastic, and may include but is not limited to wire, rod, tube, coil, braid, cable or any combination thereof.

FIG. 17 illustrates an occlusion device/system 1700 with an occlusion implant 1701 deployed outside of the distal end 113 of the delivery catheter 102. The occlusion implant 1701 comprises a distal braid 1702 and a proximal braid 1703, both shown in expanded configuration. The distal braid 1702 has a larger diameter than proximal braid 1703 in its expanded configuration. The larger distal braid 1702 has a distal tip 1704. The distal braid 1702 and the proximal braid 1703 are connected together by an intermediate internal member 1705 at a connection area 1706. The intermediate connecting member 1705 may have any suitable shape or configuration, may be made of metal or plastic, and may include but is not limited to wire, rod, tube, coil, braid, cable or any combination thereof. Both braid attachments to the intermediate internal member 1705 may be accomplished using any suitable method, including but not limited to bonding, fusing, gluing welding or soldering. The proximal braid 1702 and the distal braid 1703 may also be connected directly without using an intermediate internal connecting member 1705 (not shown). Additionally, a radiopaque marker 1707 may be positioned on the distal end of the distal braid 1702, another radiopaque marker may be positioned at the attachment member 1705 (not shown), and another radiopaque marker 1708 may be positioned on the proximal end of the proximal braid 1703. The proximal end of the proximal braid 1703 is attached to the pusher member 103 at the attachment area 115. Alternatively, one or more elongate constraining members may be extended within one or both braids, and optionally include radiopaque members (not shown).

The occlusion implant 1701 may include a plurality of braids with a variety of different dimensions, including smaller sizes, larger sizes, as well as a variety of cross-sectional configurations including but not limited to circular, non-circular and combination of both (not shown).

The occlusion implant shown and described in FIG. 17 provides an effective engaging/anchoring edifice with the first distal expanded braid 1702. When combined with the second smaller/space filling expanded braid 1703, adding a large volumetric area will promote quick blood clotting.

Expandable braids used for the occlusion implants for treatment of defects in humans require several unique characteristics, including but not limited to softness and flexibility, low profile when in the collapsed configuration, and most importantly, ability to be delivered to the treatment locations through a small profile delivery catheter. The braid(s) when delivered through a delivery catheter is in a collapsed configuration that creates radial outward forces and causes a lot of friction between the outside surface of the braid and the inner lumen of the catheter, making such delivery difficult and often time-consuming. One of the known methods in the art to reduce such friction is by providing an inner lumen of the delivery catheter with a polymer having a low friction coefficient, such as Polytetrafluoroethylene (PTFE).

Another method to further reduce such friction is by providing a braid that has as small a braid angle as possible when in the expanded configuration. Such braid with a small expanded braid angle would create lower radial outward forces and consequently less friction when the braid is delivered through the delivery catheter. There are significant technical challenges/limitations to construct a braid made of a small NiTi wire between 0.0005″-0.0010″ at an angle of less than 60 degrees. Often, braids manufactured at angles below 60 degrees are unstable, inconsistent and frequently unreliable.

The present invention provides a braid that is initially made with a primary/first outside diameter and a primary/first braid angle, and then is re-configured to a smaller secondary braid configuration having a secondary outside diameter that is smaller than the original primary/first braid diameter and has a smaller braid angle than the primary braid angle. Such braid modification may be achieved by placing the primary braid over a smaller diameter mandrel and stretching the braid, or collapse-forcing the braid along that mandrel, and fixing both ends to prevent the braid from returning to the original configuration. Fixing the braid ends may be done using a small wire and tightly looping/squeezing both ends of the braid after stretching so the braid will not re-spring to its original configuration. Such prepared braid may then be thermally re-shaped to a new secondary configuration having a smaller outside diameter and smaller braid angle.

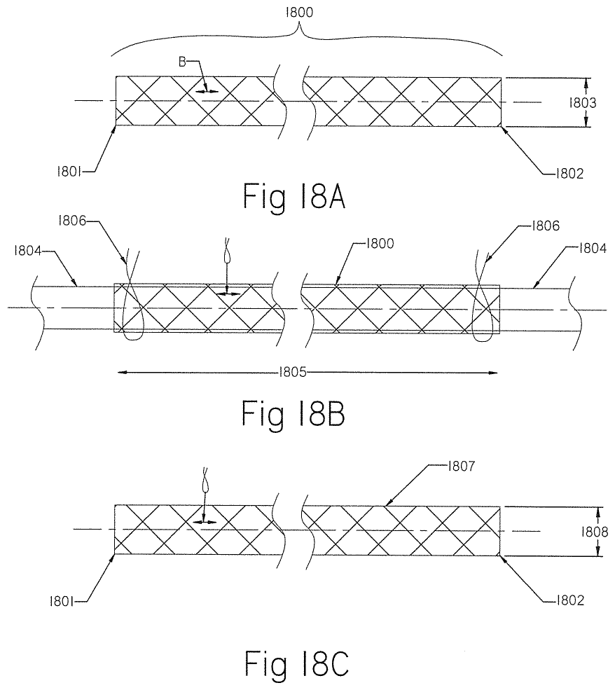

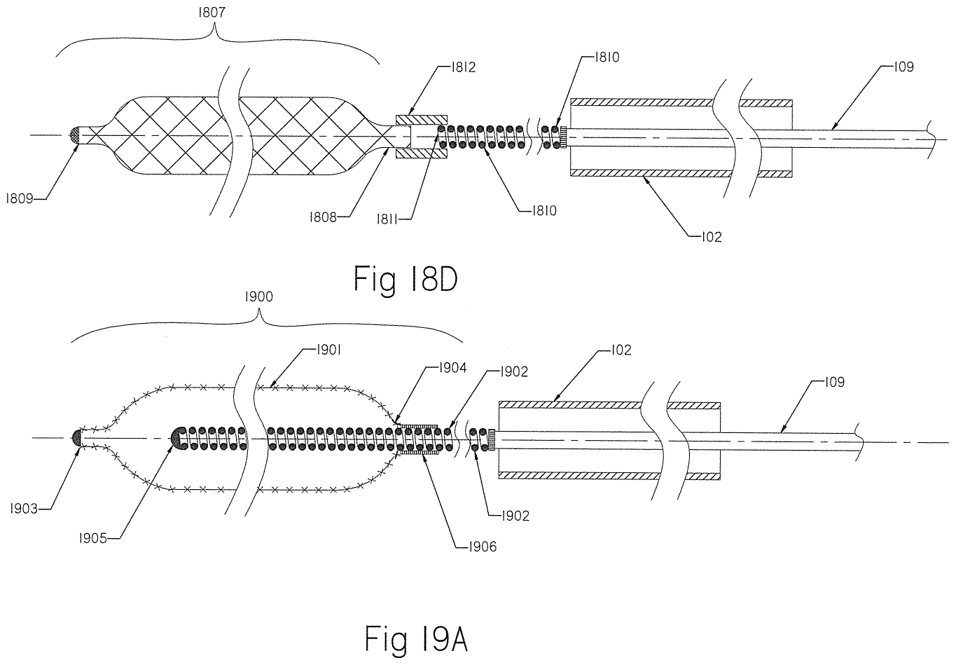

FIG. 18A shows a braid 1800 having a distal end 1801, and a proximal end 1802, and has a primary (after original manufacturing/braiding) outside diameter 1803 and a primary braid angle B. FIG. 18B shows the braid 1800 placed and stretched over the mandrel 1804 as shown by arrows 1805. The distal end 1801 of the braid 1800 is secured to the mandrel 1804 using a flexible/soft wire 1806. Since the thermal shaping of the braid 1800 is performed at a very high temperature, often exceeding 500 degrees Celsius, it is preferable to use metal or metal alloy wires for such application. The braid 1800 is stretched over the mandrel 1804 and thermally reconfigured to a secondary braid configuration 1807 having a secondary outside diameter 1808 that is smaller than primary outside diameter 1803 and has a smaller braid angles than the primary braid angle B as shown in FIG. 18C.

The secondary braid angle r should preferably be less than 60 degrees when in the expanded configuration to further reduce friction within the delivery catheter. The braid 1807 may be made in one of the following patterns: 1 over-1 under wire, 2 over-2 under wires, 1 over-2 under wires, 2 over-2 under wires, and combinations thereof. These braid configurations are well known in the art and will not be described in detail herein. Each pattern has advantages or disadvantages to achieve the braid's ability to open to the expanded configuration when released from a small delivery catheter. However, the 1 over-1 under wire pattern appears to produce the lowest friction resistance when delivered through a delivery catheter while in a collapsed configuration.

FIG. 18D shows the secondary braid 1807 from FIG. 18C having a proximal end 1808 and a distal end 1809. A helical coil 1810 having a distal end 1811 may be attached to the proximal end 1809 of the braid 1807 using an intermediate external tube member 1812 located between the proximal end 1808 of the braid 1807 and distal end 1811 of the coil 1810 to connect the braid 1806 and the coil 1810. The proximal end 1808 of the braid 1807 may be positioned inside the intermediate tube member 1812 on one end of the member 1812, and the distal end 1811 of the coil 1810 may be positioned inside the intermediate tube member 1812 on the opposite end. The intermediate external tube 1812 may be made of one of the following materials: polymer, metal, metal alloy, rubber, ceramic or any combination thereof. The proximal end 1808 of the braid 1807 and the distal end 1811 of the coil 1810 may be in contact or spaced apart. The connection area between the braid 1807 and the coil 1810 that includes the intermediate member 1812 should provide a suitable transition allowing navigation of the catheter during the access to the treatment area and the deployment of the implant.

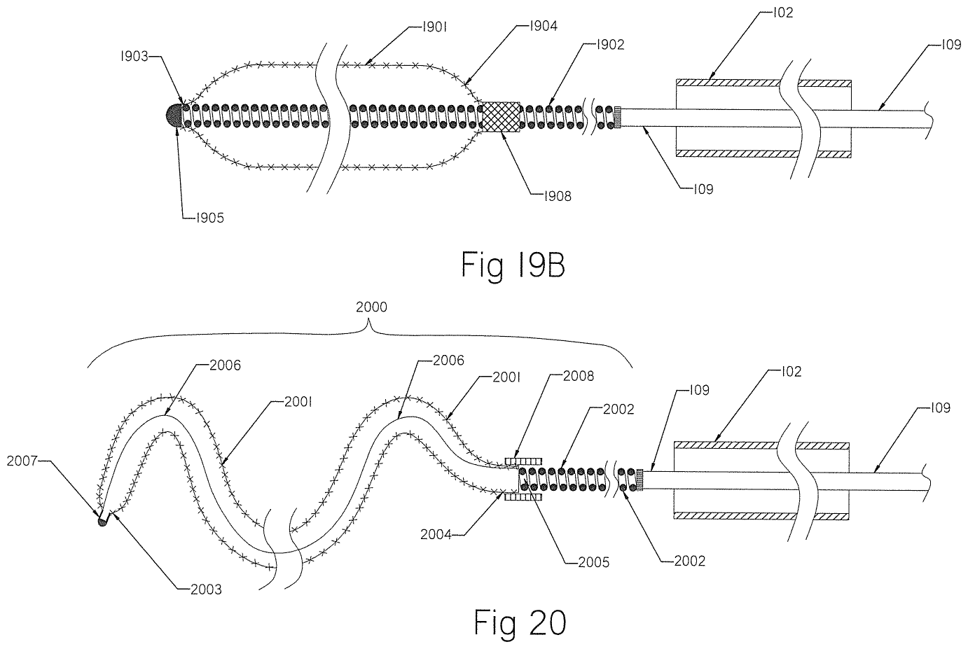

FIG. 19A shows an occlusion implant 1900 comprising a braid 1901 and a helical coil 1902. The braid 1901 has a distal end 1903 and a proximal end 1904. The coil 1902 has a distal end 1905. The coil 1902 is at least partially extended inside the braid 1901. The braid 1901 and the coil 1902 are connected together at the proximal end 1904 of the braid 1901 at a connection area 1906. The distal end 1905 of the coil 1902 is freely extended inside the braid 1901 and is unattached. Since the coil 1902 is extended internally along the braid 1901, the braid 1901 and the coil 1802 traverse concomitant bends when they are pushed through and retrieved back into the delivery catheter (not shown).

FIG. 19B shows the occlusion implant 1900 having the helical coil 1902 fully extended inside/through the braid 1901. The distal end 1903 of the braid 1901 and distal end 1905 of the coil 1902 are connected together. The proximal end 1904 of the braid 1901 and the coil 1902 are connected together at the connection area 1908. The coil 1902 is fully extended internally along the braid 1901, and the coil 1902 and the braid 1901 traverse concomitant bends when they pushed through and retrieved back into the delivery catheter (not shown). The proximal end 1904 of the braid 1901 may also be un-affixed to the coil 1902 in a free-floating fashion and can be re-positioned back and forth along the coil 1902 as needed (not shown).

The occlusion devices/system 1900 may be comprised of two separate coils: one coil located proximal to the braid, and one located inside the braid (not shown). Such coil(s) may have one of the following configurations: straight, not heat pre-shaped, heat pre-shaped and a combination thereof.

FIG. 20 shows an occlusion implant 2000 comprising a braid 2001 and a helical coil 2002. The braid 2001 has a distal end 2003 and a proximal end 2004. The helical coil 2002 has a distal end 2005. At least one constraining member 2006 is extended longitudinally through the braid 2001 and attached to the distal end 2003 of the braid 2001 and to the proximal end 2004 of the braid 2001 at the area 2007. The constraining member 2006 may be configured to have a pre-set expanded shape when released from the delivery catheter. The wavy shape of the constraining member 2006 is shown for reference only.

The constraining member 2006 may be pre-shaped by heat to any desired configuration/shape appropriate for treating endovascular and non-endovascular defects. The braid 2001 is suitable to assume a pre-set expanded shape/configuration of the constraining member 2006 when pushed outside the delivery catheter. The braid 2001 and constraining member 2006 may traverse concomitant bends when pushed through and retrieved back into the delivery catheter (not shown). The proximal end 2004 of the braid 2001 is connected to the distal end 2005 of the helical coil 2002 using an intermediate member 2008.

The constraining member 2006 and the braid 2001 may also both have thermally pre-shaped configurations, and both may assume a similar configuration after release from the delivery catheter. The constraining member 2006 is made of a metal or metal alloy, preferably Nitinol.

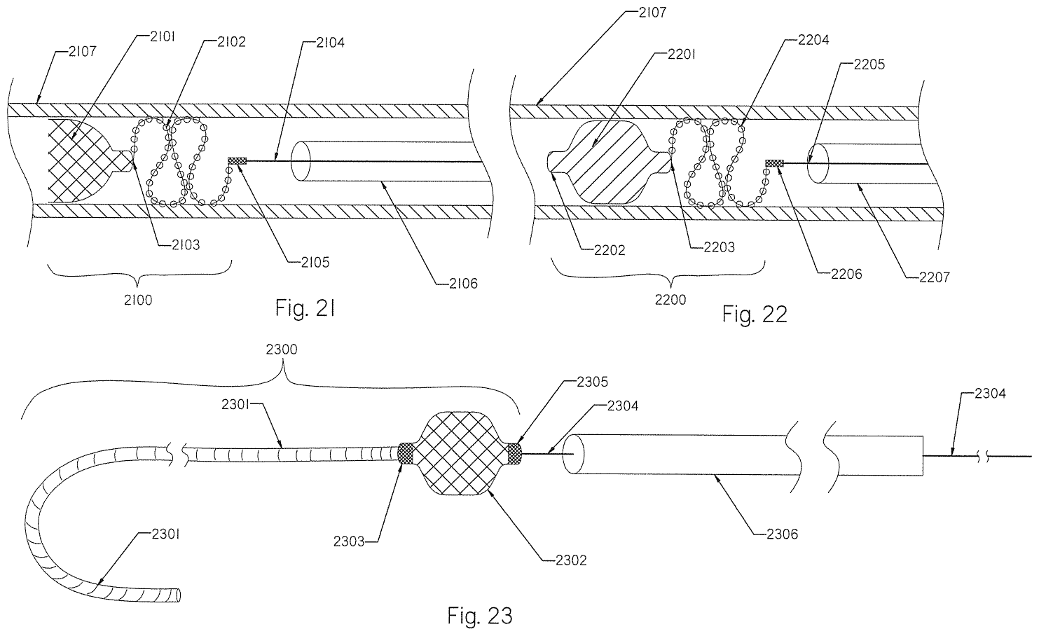

FIG. 21 illustrates the TED device or occlusion implant 2100 of the present invention comprising an open-ended expandable braid 2101with a coil 2102 attached at an attachment area 2103. The implant 2100 is attached to the pusher member 2104 at an attachment area 2105 and is delivered via the delivery catheter 2106 to the vessel 2107. The occlusion implant 2100 is detached from the pusher member 2103 and deployed inside the vessel 2107 to block blood flow and occlude the vessel 2107.

While the expandable braid 2101 is anchoring the vessel 2107, deployment of the coil 2102 provides an additional barrier to mitigate forces from blood flow to further slow blood flow and occlude the vessel 2107.

The pusher member 2104 traverses concomitant bends as the delivery catheter 2106 during its delivery to the treatment location inside the vessel 2107. Upon deployment of the occlusion implant 2100 into the vessel 2107, the open-ended braid 2101 expands/opens into an expanded configuration assuming a pre-set shape and anchoring into the wall of the vessel 2107. The helical coil 2102 further fills the space behind the braid 2101.

The occlusion implant 2100 may also be withdrawn and collapsed back into the delivery catheter 2106 in case the deployment of the implant 2100 into the vessel 2107 is not satisfactory or needs repositioning. The placement of the occlusion implant 2100 inside the vessel 2107 may be repeated multiple times until a correct deployment position is achieved. When the braid 2101 expands inside the vessel 2107 and reaches an expanded configuration and pre-shaped contour, it begins to occupy most of the space inside the vessel 2107, providing an engagement structure for the deployment of the helical coil 2102 and to further mitigate blood pressure on the braid 2101 and facilitating the clotting of blood. Once the occlusion implant 2100 is positioned in the desired location within the vessel 2107, the occlusion implant 2100 is disconnected (detached) from the pusher member 2103 and blood clotting of the vessel 2107 begins.

FIG. 22 shows an alternative occlusion implant 2200 inside a vessel 2107 having a braid 2201 with a distal closed end 2202 and a proximal closed end 2203, with a coil 2204 attached to the proximal closed end 2203. The occlusion implant 2200 is attached to the pusher member 2205 at the attachment area 2206 shown here partially with the delivery catheter 2207. Such occlusion implant configurations are deployed and function in the same manner as the occlusion implant 2100 described above, and may be used for the same clinical applications. It is important to mention that braids of the occlusion implants with closed ends may be better suitable for vessel closure in the brain, where smaller and more fragile vessels are more frequently found.

Alternatively, the occlusion implant 2100 in FIG. 21 may comprise the braid 2101 only (not shown) and the occlusion implant 2200 in FIG. 22 may comprise the braid 2201 only (not shown). The braid 2101 in FIG. 21 and the braid 2201 in FIG. 22 may be used alone and deployed into the vessel 2107 without proximally attached coils (not shown). Longer braids may be used in such scenarios to compensate and/or substitute the mass/volume of the previously attached coils.

FIG. 23 shows an occlusion implant 2300 comprising a helical coil 2301 attached at a proximal end to an expandable braid 2302 at an attachment area 2303. The braid 2302 is attached at its proximal end to a pusher member 2304 at an attachment area 2305, and is shown outside the delivery catheter 2306.

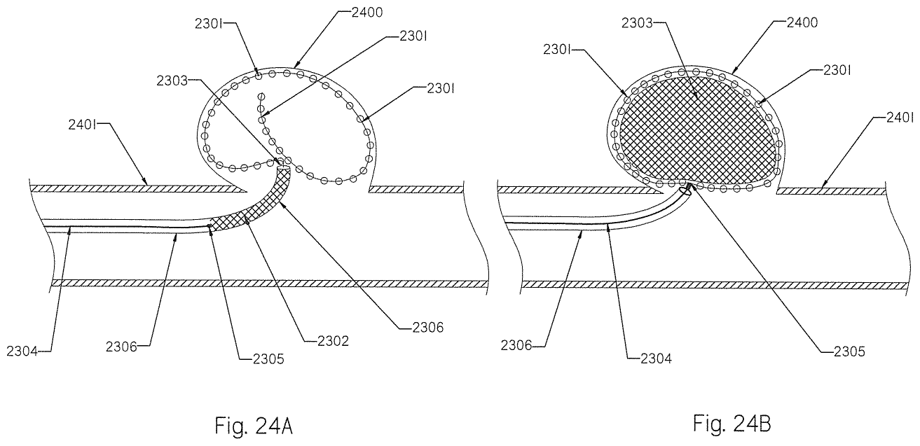

FIG. 24A shows the occlusion implant 2300 of FIG. 23 delivered via the delivery catheter 2306 into the aneurysm sac 2400 of a vessel 2401. The helical coil 2301 of the occlusion implant 2300 is deployed inside the aneurysm sac 2400 first. The helical coil 2301 is deployed inside the aneurysm sac 2400 by pushing the entire occlusion implant 2300 using the pusher member 2304. The deployed coil 2301 within the aneurysm sac 2400 creates loops and anchors the structure within and around the inner wall of the aneurysm sac 2400.

Alternatively, the occlusion implant 2300 in FIG. 23 may be open ended on the distal end having the proximal end of the coil 2301 attached at the proximal end of the braid 2302 at the attachment point 2305 (not shown).

Alternatively, the occlusion implant 2300 in FIGS. 23, 24A and 24B may comprise the braid 2302 only. The braid 2302 may be used alone and deployed into the aneurysm 2400 without proximally attached coils as previously described

After the distal coil 2301 is deployed and assumes a pre-set configuration within the aneurysm sac 2400, the braid 2302 is deployed. The braid 2302 expands and assumes a pre-set shape as shown in FIG. 24B filling the aneurysm sac 2400 with its braided/meshed structure. The implant 2300 may also be removed or withdrawn and collapsed back into the delivery catheter 2306 if the position of the occlusion implant 2300 (including the coil 2301 and/or the braid 2302) within the aneurysm sac 2400 is not satisfactory. The helical coil 2301 provides an engagement structure for the expandable braid 2302, thereby providing additional foundation for stability of the braid 2302 and preventing deconfiguration or collapse of the braid 2302. After deployment, the braid 2302 begins to occupy a greater space within the aneurysm sac 2400, and consequently fills the space within the aneurysm sac 2300. The deployed occlusion implant 2300 prevents blood penetration inside the aneurysm sac 2400 and promotes blood clotting of any blood inside the aneurysm sac 2400.

While the braid shown in the FIG. 23 and FIGS. 24A & 24B has a closed configuration with both ends closed, in alternative configurations for the occlusion implant, the braid may be open ended with one end open (not shown).

When the delivery catheter 2306 traverses bends and anatomical curves to access the aneurysm sac 2400, the occlusion implant 2300 in its collapsed configuration traverses concomitant bends as the delivery catheter 2306.

The combination of two dissimilar metals of the occlusion implant 2300, such as the coil 2301 made of Platinum and the braid 2302 made mostly of NiTi alloy, placed in an aqueous environment within the suck of the aneurysm 2400 will create a potential difference between the two metals. The greater the electrical potential between two metals, the more likely a current will be generated. Platinum is one of the lowest in the electro-potential series for metals and as such will be least likely to corrode relative to the other metal such for example NiTi.

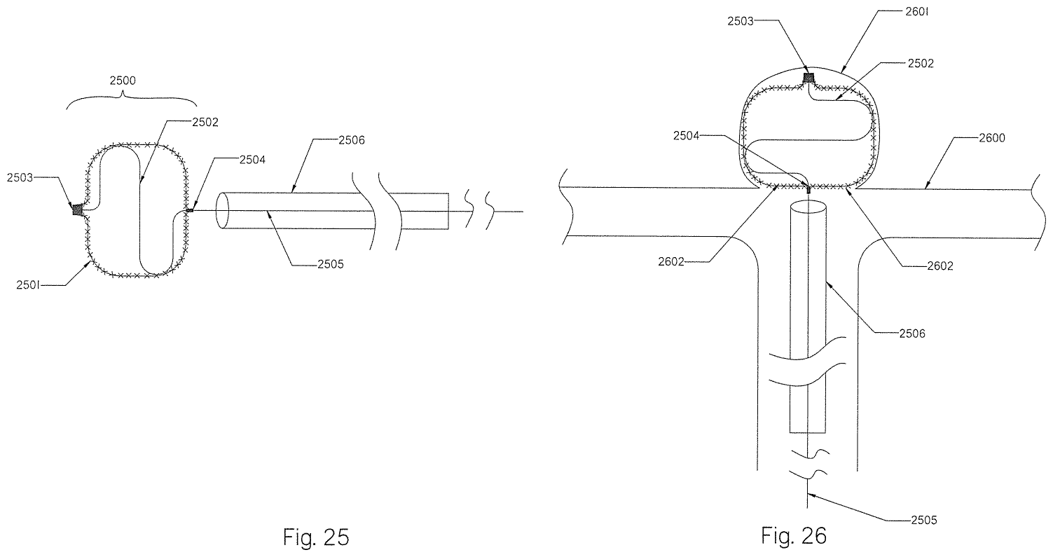

FIG. 25 illustrates an MEF device 2500 comprising an expandable braid 2501 and an elongate constraining member 2502. The expandable braid 2501 is connected to the constraining member 2502 at a distal attachment area 2503 and at a proximal attachment area 2504 using conventional attachment methods, including but not limited to bonding, welding, crimping or heat fusing. The MEF device 2500 is connected to the pushing member 2505 and delivered via the delivery catheter 2506. The elongate constraining member 2502 may have a variable stiffness along its length, being stiffer distally and more flexible proximally, and vice versa.

The constraining member 2502 may be made of a single wire, multiple wires, strands, coils, tubes, polymer rod, knit, woven, braid and have several configurations suitable to internally support the braid 2501, including but not limited to: straight, tubular, bent, coiled, helical, sinusoidal, wave, closed basket, open basket shaped fingers, open mash, closed mesh or any combination thereof. Such elongate constraining member 2502 may be made of metals, alloys, shape memory material (e.g., Nitinol), cobalt-chromium alloys, Platinum, Platinum-Iridium alloys, polymers (e.g., Nylon, Polyester, etc.), Nitinol with platinum core, or combination thereof.

The constraining member 2502 is extended inside the braid 2501and may be attached either to the distal attachment area 2503 or to the proximal attachment area 2504, or to both attachment areas 2503 and 2504 as shown in FIG. 25. The constraining member 2502 and the braid 2501 may also both have thermally pre-shaped configurations, and both may assume a similar configuration after release from the delivery catheter 2506. A pre-set expanded shape/configuration of the constraining member 2502 when pushed outside the delivery catheter 2506 may further help the braid 2501 to assume its desirable expanded shape.

The elongate constraining member 2502 may also enhance the radiopacity of the MEF device, by virtue of its composition. The constraining member 2502 may also comprise a bioabsorbable material and be dissolved after time.

The constraining member 2502 provides additional internal forces within the braid 2501 to prevent the expanded braid 2501 from squeezing, crushing, collapsing, folding after deployment at the treatment area as shown in FIG. 26.

FIG. 26 illustrates the MEF device 2500 of FIG. 25 deployed inside an aneurysm sac 2601 of the vessel 2600. The constraining member 2502 is fully expanded to its pre-shaped configuration. The pre-shaped constraining member 2502 may assume any desirable configuration within the braid 2501, including but not limited to linier sinusoidal shape, helical shape, straight bent shape and any other desirable shape configure to provide internal support for the braid 2501.

After deployment of the MEF device 2500 inside the sac 2601, blood flow dynamics within the vessel 2600 will cause a pressure from forces created by blood flow and pulsation at the neck of the sac 2601, thereby stressing the braid portions 2602 of the braid 2501 and potentially causing the braid 2501 to experience squeezing, crushing, collapsing or folding. The constraining member 2502 is constructed and designed to prevent, reduce or minimize such deformations by the braid 2501, thereby limiting blood flow from the vessel 2600 into the aneurysm sac 2602 (aneurysm recanalization) and consequently, preventing blood bleeding outside of the aneurysm sac 2601, and preventing aneurysm sac 2601 from rapture and hemorrhagic stroke.

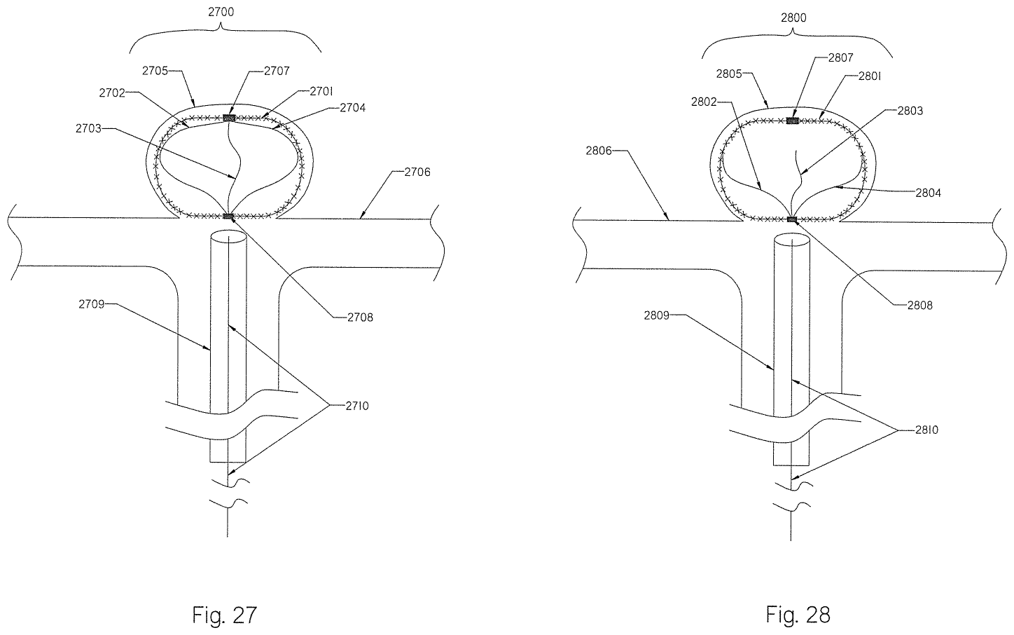

FIG. 27 illustrates an alternative configuration of the MEF device 2700 comprising the braid 2701 and three constraining members 2702, 2703 and 2704 deployed inside the aneurysm sac 2705 of the vessel 2706. The braid 2701 and constraining members 2702, 2703, 2704 are attached together at the distal attachment point 2707 and at the proximal attachment point 2708. The MEF device 2700 is delivered to the aneurysm sac 2705 using the delivery catheter 2709 and the pushing member 2710.

FIG. 28 illustrates another alternative configuration of the MEF device 2800 comprising the braid 2801 and three constraining members 2802, 2803 and 2804 deployed inside the aneurysm sac 2805 of the vessel 2806. The braid 2801 and one end of each of the constraining members 2802, 2803, 2804 is attached to the proximal attachment point 2808. Opposite ends of the constraining member 2802, 2803 and 2804 are freely located inside the braid 2801. The MEF device 2800 is delivered to the aneurysm sac 2805 using the delivery catheter 2809 and pushing member 2810. Alternatively, constraining members 2802, 2803 and 2804 may be attached at one end to the braid 2801 at the distal attachment end 2807 having other ends not attached to the braid 2801, and freely positioned inside the braid 2801(not shown).

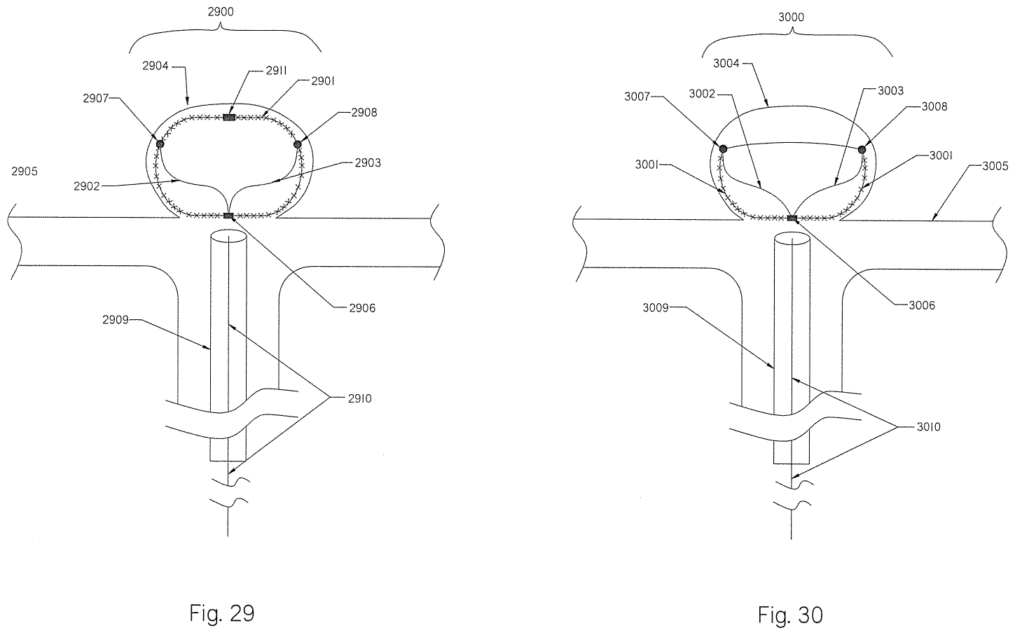

FIG. 29 illustrates another alternative configuration of the MEF device 2900 comprising the braid 2901 and two constraining members 2902 and 2903 deployed inside the aneurysm sac 2904 of the vessel 2905. The braid 2901 and one end of each of the constraining members 2902 and 2903 are attached to together at the proximal attachment point 2906. Opposite ends of the constraining member 2902 and 2903 are attached to the braid 2801 at the attachment areas 2907 and 2908 inside the braid 2901. The MEF device 2900 is delivered to the aneurysm sac 2904 using the delivery catheter 2909 and pushing member 2910. Alternatively, one end of constraining members 2902 and 2903 may be attached to the braid 2901 at the distal attachment point 2911 and having the other ends attached to inside the braid 2901 (not shown).

FIG. 30 illustrates another alternative configuration of the MEF device 3000 comprising the open-ended braid 3001 and two constraining member 3002 and 3003 deployed inside the aneurysm sac 3004 of the vessel 3005. The braid 3001 and one end of each of the constraining members 3002 and 3003 are attached together at the proximal attachment point 3006. Opposite ends of the constraining member 3002 and 3003 are attached to the braid 3001 at the attachment areas 3007 and 3008. The MEF device 3000 is delivered to the aneurysm sac 3004 using the delivery catheter 3009 and pushing member 3010.

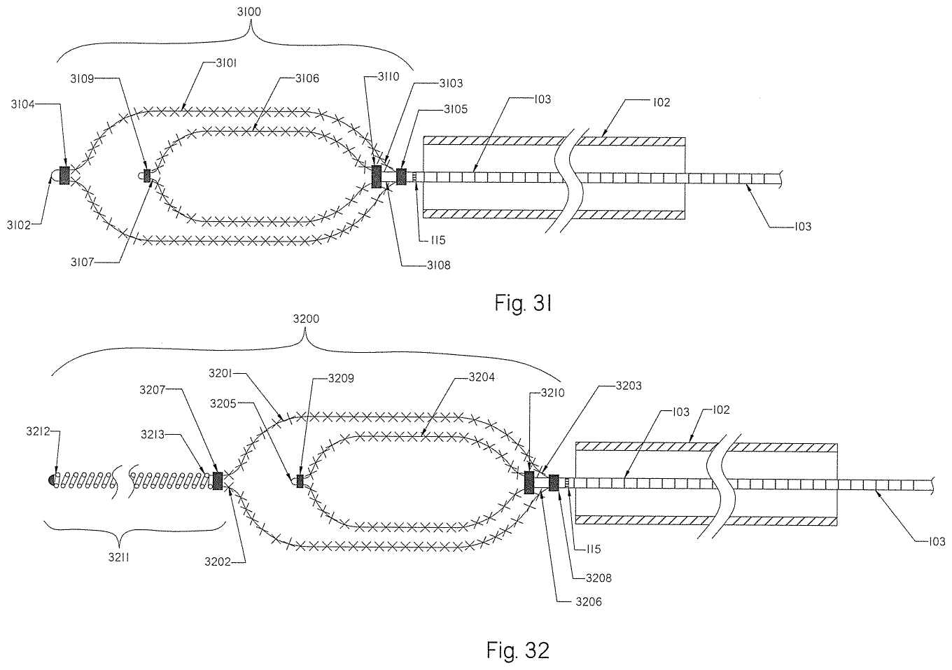

FIG. 31 shows an occlusion implant 3100 that includes an outer braid 3101 having a distal end 3102 and a proximal end 3103. One radiopaque marker 3104 may be located on the distal end 3102 of the braid 3101 and another radiopaque marker 3105 may be located on the proximal end 3103 of the braid 3101. The proximal end 3103 of the braid 3101 is attached to the pusher wire 103 at the attachment area 115 located on the delivery catheter 102.

A second inner braid 3106 has a distal end 3107 and a proximal end 3108, and is located inside the outer braid 3101. One radiopaque marker 3109 may be located on the distal end 3107 of the braid 3106 and another radiopaque marker 3110 may be located on the proximal end 3108 of the braid 3106. The proximal end 3108 of the braid 3106 and the proximal end 3103 of the braid 3101are attached together at the attachment area 115. Alternatively, the proximal end 3108 of the inner braid 3106 may be attached to the proximal end 3103 of the outer braid 3101.

The distal end 3102 and the proximal end 3101 are end points of the outer braid 3101, and the distal end 3107 and the proximal end 3108 are end points of the inner braid 3106 located inside the braid 3101. These endpoints prevent the distal and proximal ends of the outer braid 3101, and inner braid 3106, from expanding when deployed from the delivery catheter 102.

The occlusion implant 3100 can be deployed by a delivery catheter 102, and may extend longitudinally within the delivery catheter 102 and may be configured to be pushed through and out of the delivery catheter 102, and retrieved back into the distal end of the delivery catheter 102 using a pushing member 103. The occlusion implant 3100 may at least be partially expanded to a larger volumetric area when pushed out of delivery catheter 102. This partial expansion may include expansion of the outer braid 3101 and expansion of the inner braid 3106 inside the outer braid 3101. The outer braid 3101 and the inner braid 3106 may be configured to have pre-set expanded shapes when released from the delivery catheter 102.

The distal end 3107 of the inner braid 3106 is free floating inside the outer braid 3101. The length of the inner braid 3106 may vary and can be selected as desired. In one embodiment, the length of the inner braid 3106 may be 5-10% of the overall length of the outer braid 3101, and in another embodiment, it may be 5-100% of the overall length of the outer braid 3101. If desired, the distal end 3107 of the braid 3106 may be attached to the outer braid 3101 at any suitable location inside the outer braid 3101.

The outer braid 3101and the inner braid 3106 may assume pre-set concomitant expanded shapes, or different expanded shapes, when pushed outside the delivery catheter 102. The inner braid 3106 can perform functions that may include, but are not limited to, support for the outer braid 3101, preventing the outer braid 3101 from collapsing or flattening, and facilitating expansion of the outer braid 3101.

FIG. 32 shows an occlusion implant 3200 that includes an outer braid 3201 having a distal end 3202 and a proximal end 3203, and a second inner braid 3204 located inside the outer braid 3201 and having a distal end 3205 and a proximal end 3206. The proximal end 3206 of the inner braid 3204 and the proximal end 3203 of the outer braid 3201 are attached together at an attachment area 115. The outer braid 3201 has a distal radiopaque marker 3207 and a proximal radiopaque marker 3208. The inner braid 3204 has a distal marker 3209 and a proximal marker 3210. A coil 3211 has a distal end 3212 and a proximal end 3213 which is attached to the distal end 3202 of the outer braid.

This hybrid structure occlusion implant 3200 that has dual braids (the outer braid 3201 and the inner braid 3204) and the coil 3211 is attached to the pusher member 103 located inside the delivery catheter 102 at the attachment area 115. The distal coil 3211 provides a lead or guide when entering a treatment area. Deployment of a distal coil 3211 from the delivery catheter 102 first into the treatment area will form a pre-shaped anchoring structure around the treatment area and provides a support and stability for delivery of the dual-braids to fill out and pack the treatment area, and to create a quick and reliable occlusion. As with the other occlusion implants of the present invention, the dual-braids with the distally attached coil 3200 may traverse concomitant bends as the delivery catheter 102 when pushed through and retrieved back into the delivery catheter 102.

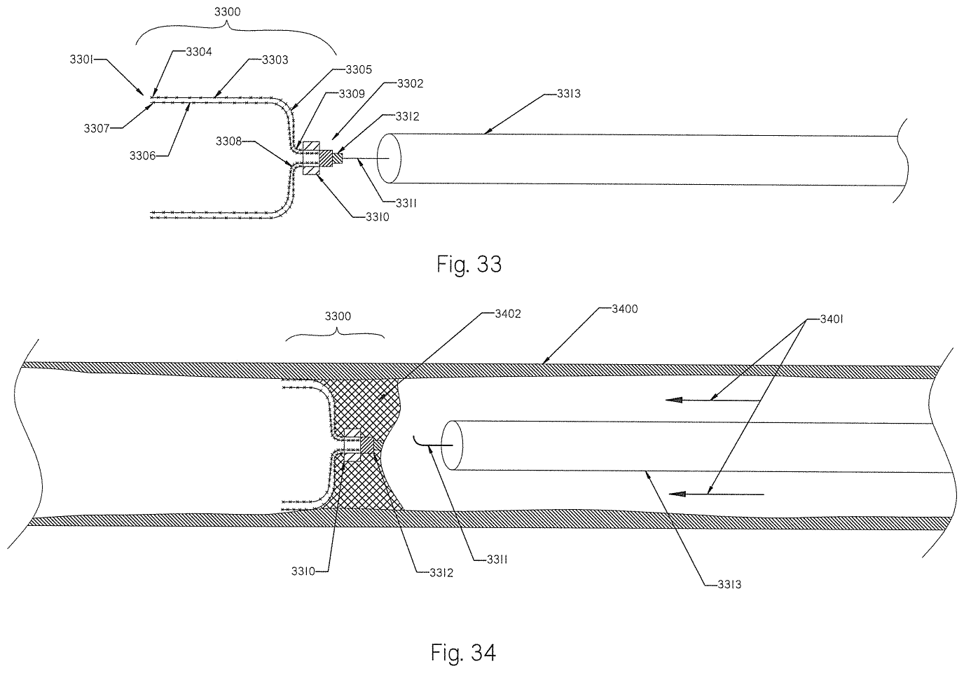

FIG. 33 shows an embolization plug assembly that has a plug 3300 having a distal end 3301 and a proximal end 3302. The plug 3300 comprises an outer braid 3303 having a distal end 3304 and the proximal end 3305, and an inner braid 3306 having a distal end 3307 and a proximal end 3308. The distal end 3301 of the plug 3300 is open ended. Therefore, the distal end 3304 of the outer braid 3303 and the distal end 3307 of the inner braid 3306 are also open ended.

The opened distal end 3304 of the outer braid 3303 and the opened distal end 3307 of the inner braid 3306 can be aligned to terminate along the same planar line. In an alternative embodiment, the distal end 3304 of the outer braid 3303 may be extended more distally than the distal end 3307 of the inner braid 3306 (not shown). In yet another alternative embodiment, the distal end 3307 of the inner braid 3306 may be extended more distally than the distal end 3304 of the outer braid 3303 (not shown). 214. The proximal-most end of the proximal portion 3308 of the inner braid 3306 is in a collapsed, closed configuration, and is extended inside of the proximal-most end of the proximal portion 3305 of the outer braid 3303 which is also in a collapsed, closed configuration. Both of these proximal ends of the inner and outer braids are attached together at the area 3309 via attachment mechanisms that may include, but are not limited to, glue, bonding, welding, soldering, fusing or any similar suitable attachment methods.

A radiopaque marker 3310 may be located on the proximal end 3302 of the plug 3300. The proximal end 3302 of the plug 3300 is attached to a pusher wire 3311 via a detachable attachment mechanism 3312. The detachable attachment mechanism 3312 may include, but is not limited to, a mechanical detachment, or electrolytic or any other known types of detachable attachment mechanisms.

The plug 3300 and the pusher wire 3311 are located inside a delivery catheter 3313 when the plug 3300 is delivered to the treatment area. The plug 3300 is preferably detached from the pusher wire 3311 outside of the delivery catheter 3313.

A delivery catheter 3313 is used to deliver the plug 3300 to the treatment area. The delivery catheter 3313 is configured to push the plug 3300 through and out of the delivery catheter 3313, and to retrieve the plug 3300 back into the distal end of the delivery catheter 3313 using the pushing wire 3311 if necessary. The plug 3300 may be partially expanded to a larger volumetric area when pushed out of the delivery catheter 3313. This partial expansion may include expansion of the outer braid 3303 and expansion of the inner braid 3306.

The plug 3300, including the outer braid 3303 and the inner braid 3306, may be configured to have pre-set expanded shapes when released from the delivery catheter 3313.

The plug 3300 may be provided in one of several configurations, including but not limited to, circular, bulbous, ball-shaped, onion-shaped, oval, flat, rectangular, tear-shaped, twist-shaped, non-circular, curved shaped, and can be three-dimensional, or assume any random or non-linear shape.

The distal end 3307 of the inner braid 3306 can be free floating inside the outer braid 3303. The length of the inner braid 3306 may be the same as the outer braid. Alternatively, the inner braid 3306 may be longer or shorter than the outer braid (not shown). The distal end 3307 of the inner braid 3306 may be attached to the outer braid 3303 at any location inside the outer braid 3303 if needed (not shown).

The outer braid 3303 and the inner braid 3106 may assume pre-set concomitant expanded shapes when pushed outside the delivery catheter 3313. The inner braid 3306 can perform functions that may include, but are not limited to, support for the outer braid 3303, preventing the outer braid 3303 from collapsing or flattening, facilitating expansion of the outer braid 3303 and any required function.

FIG. 34 shows the plug 3300 of FIG. 33 deployed inside a blood vessel 3400. The deployment of the plug 3300 may be directed along the blood flow as shown by arrows 3401, against the flow of blood, from the arterial side or from a venous site.

The hybrid structure of the plug 3300 that comprises two braids provides a reliable anchoring structure for the plug 3300 against the vessel 3400 to prevent the plug 3300 from re-positioning within the vessel 3400, so that the plug 3300 can instantaneously limit blood flow and create a rapid occlusion 3402 inside the vessel 3400.

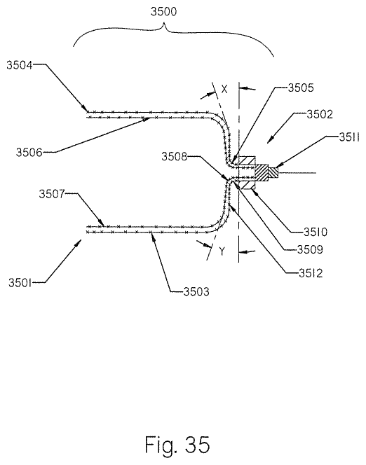

FIG. 35 shows a more detailed view of a plug 3500. The plug 3500 can be the same as the plug 3300 in FIG. 33, and even though different numeral designations are being used, it will be understood that the plugs 3300 and 3500 can be the same. The plug 3500 has a distal end 3501 and a proximal end 3502. The plug 3500 comprises two braids: an outer braid 3503 having a distal end 3504 and a proximal end 3505, and an inner braid 3506 having a distal end 3507 and a proximal end 3508. The distal end 3501 of the plug 3500 is in an expanded configuration and open ended. Accordingly, the distal end 3504 of the outer braid 3503 and the distal end 3507 of the inner braid 3506 are also in expanded configurations and are open ended. The inner braid 3506 is freely positioned inside the outer braid 3503.

The proximal end 3505 of the outer braid 3503 and the proximal end 3508 of the inner braid 3506 are both in a collapsed configuration. The proximal end 3508 of the inner braid 3506 is positioned inside the proximal end 3505 of the outer braid 3503. The two proximal ends may be attached together at the area 3509 and may also be constricted with a marker band 3510. A detachable attachment mechanism 3511is located on the very proximal end 3502 of the implant 3500. The detachable attachment mechanism 3511 may be provided in the form of any suitable detachable attachment mechanism, including a mechanical connection, or electrolytic or other detachments.

The inner braid 3506 may be configured to facilitate expansion of the outer braid 3503 and to assist the outer braid 3506 in anchoring at the treatment area, and to limit blood flow through the plug to create a quick occlusion at the treatment area upon deployment.

The inner braid 3506 and the outer braid 3503 may be formed from a plurality of wire strands having a dimension that is between about 0.0003 inches and about 0.010 inches, and wherein the wire strands are made of one of the following materials: metals, alloys, shape memory material (e.g., Nitinol), cobalt-chromium alloys, Platinum, Nitinol-Platinum alloys, Platinum-Iridium alloys, polymers (e.g., Nylon, Polyester, etc.) or any combination thereof, and wherein the braid includes strands of the same dimensions or of different dimensions that are braided using a circular wire, an oval wire, a flat wire, or any other suitable wire configuration.

The overall length of the plug 3500 plays a very important role in clinical applications; shorter plugs are more desirable and more clinically suitable than longer plugs. Shorter plugs provide more precise deployment and avoid occluding larger areas that may not be clinically beneficial. 229. To reduce the length of the plug 3500, a plug shoulder or tapered portion 3512 should have a steep angulation. To achieve a steep angulation of the tapered portion 3512, the tapered angle X of the outer braid 3503 should be tapered to 30-80 degrees. This angle X is defined as being with respect to an axis that is perpendicular to the longitudinal axis of the plug 3500. Such braid angulation may be achieved by a variety of heat treatments and by setting a pre-set expanded shape of the outer braid 3503.

The tapered angle Y of the inner braid 3506 may have any desired angulation to perform its functions, and to be consistent with the requirements of the outer braid 3503. This angle Y is defined as being with respect to an axis that is perpendicular to the longitudinal axis of the plug 3500.

The outer braid 3503 may have a primary braid configuration having a primary outside diameter and a primary braid angle after it is manufactured. This configuration for the outer braid 3503 may be further reconfigured to a secondary braid configuration having a secondary outside diameter and shape that has a different braid angle than the primary braid angle.

A dual braid structure of the plug 3500 limits blood flow therethrough when the plug 3500 is deployed at the treatment area, and creates a rapid occlusion. While the plug 3500 shown in FIG. 35 is made of two braids, a greater number of braids (e.g., 2, 3, 4, or more) may be used for alternative plug configurations (not shown).

The present invention includes detailed descriptions of braids, and the expandable braids of the present invention include tubular configurations, oval, bulbous, ball-shaped, onion-shaped resembling onion, square, rectangular, irregular/non-symmetrical shapes and any combination thereof. The expandable braid(s) structure may have at least a first braid portion and/or a second braid portion coupled together to helical coils located on the distal end of the braid, between braids or on the proximal end of the braid. The expandable braid of the present invention may also include at least one internal constraining member and/or additional one or more braids. The expandable braids may be linearly aligned along the entire implant or may also be out of linear alignment with the implant. The occlusion implants may include helical coils and braids having different outside dimensions and multiple configurations of the constraining member(s). While the present invention describes occlusion implants having one or more components or parts, any combination of these components an any order are incorporated in the present invention as well. Also, reducing some components or parts of the occlusion implants (i.e., removing the coil 2002 in FIG. 20 and attaching the braid 2001 having the constraining member 2006 to the pusher member 109 at the attachment area 2005) is covered by the scope of the present invention

The number of constraining members in FIGS. 25, 26, 27, 28, 29 and 30 are exemplary. According to the present invention, there can be anywhere from one constraining member to multiple number of constraining members, as desired.

The occlusion implant devices combining two different metals implanted in the sac of the aneurysm will be in an aqueous-like environment where the platinum component of the coil will be the cathode relative to the anodic NiTi braid/mesh. The electrical potential will generate a localized low current between the two metals. The charge generated will create a charged surface on the material, also known as galvanic corrosion. The presence of two dissimilar metals allows for a more permanent charge on the surface of the metal and would stimulate electro thrombosis long enough for the clot to form and mature. The longer-term dissolution of the anodic material would ultimately contribute to a reduction in mass effect and permit steady shrinkage of larger aneurysms with time.

Alternatively, the surface of the TED, the MEF, and the braid inside braid devices (with or without attached coils) may be at least partially covered with an external or internal coating to prevent blood from penetrating inside the braid when deployed at the treatment area. Such coating may include but is not limited to coatings previously described, and serve to limit the blood penetration inside the TED/MEF after deployment. Minimizing blood penetration inside the braid may prevent collapsing, deformation or relocation of the braid structure after blood inside the braid forms clots.

The present invention describes devices and methods for treatment of endovascular defects. However, it is intended that the scope of the present invention should not be limited by the particular disease but should include any and all of these devices and methods that are suitable to treat other non-endovascular defects.

Occlusion implants of the present invention are not limited to helically wound coils, and can include random wound coils, coils wound within coils, braids, and braids within braids.

While this specification includes detailed descriptions of expandable braids, the braids of the present invention include tubular configurations, oval, bulbous, ball-shaped, onion-shaped resembling onion, square, rectangular, irregular/non-symmetrical shapes and any combination thereof. The expandable braid(s) structure may have at least a first braid portion and a second braid portion coupled together or to helical coils located on the distal end of the braid, between braids or on the proximal end of the braid. The expandable braids may be linearly aligned along the entire implant or may also be out of linear alignment with the implant. The occlusion implants may include helical coils and braids having different outside dimensions.

Braids of the present invention may also include a woven mesh with variably sized apertures (openings or pores) with a particular porosity or pore density. The expandable braids of the present invention may have sections of mesh or braid having variations in density of the filaments and may include portions or bands of densely spaced filaments (i.e., lower porosity) spaced by portions or bands that are less dense (i.e., higher porosity). The less dense braid portion can have larger openings in the braid, while the denser braid portion can have smaller openings in the braid. The first and second portions of the expandable braid can be discrete structures or can be portion(s) of a unitary or monolithically constructed implant.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Thus, it is intended that the scope of the present invention herein disclosed should not be limited by the particular disclosed embodiments described above but should be determined only by a fair reading of the claims that follow.

Elements or components shown with any embodiment herein are exemplary for the specific embodiment and may be used on or in combination with other embodiments disclosed herein. The invention is susceptible to various modifications and alternative forms and should not be limited to the particular forms or methods disclosed. To the contrary, the invention is to cover all modifications, equivalents and alternatives thereof.

Some scientific and theoretical considerations have been introduced for assessing how these therapeutic methods and devices are effective; these considerations have been provided for providing an understanding of the invention only and have no relevance to or bearing on claims made to this invention.

1. A plug assembly for occluding endovascular and non-endovascular locations in the body, comprising:
a delivery catheter having a distal end, a proximal end and a lumen;
a pusher wire extending through the lumen of the delivery catheter;
an elongate detachable plug having a distal end and a proximal end, wherein the plug extends longitudinally within the lumen and is configured to be pushed through and out of the lumen and retrieved back into the distal end of the delivery catheter using the pusher wire; wherein the plug partially expands to a larger size when pushed out of the delivery catheter, and wherein the plug comprises an outer expandable braid and an inner expandable braid that is located inside the outer expandable braid, and
wherein the distal end of the plug is open ended.

2. The plug of claim 1, wherein the plug has a pre-set expanded cross-sectional shape.

3. The plug assembly of claim 1, wherein the inner braid and the outer braid are formed from a plurality of wire strands made of Nitinol and Platinum alloy.

4. The plug assembly of claim 1, wherein the inner braid and the outer braid are formed from a plurality of wire strands made of a braid of Nitinol wire and Platinum wire.

5. The plug assembly of claim 1, wherein the outer braid is configured to have a pre-set expanded shape when released from the delivery catheter.

6. The plug assembly of claim 1, wherein the inner braid is configured to have a pre-set expanded shape when released from the delivery catheter.

7. A plug for occluding endovascular and non-endovascular locations comprising:
a multiple braid plug having a distal end and a tapered proximal end, and also including an inner braid that has a proximal end, and an outer braid that has a proximal end, the plug defining a longitudinal axis;
wherein the distal end of the plug is open ended, the proximal end of the plug has a collapsed configuration, and the proximal end of the inner braid is attached to the proximal end of the outer braid, wherein the distal end of the inner braid is free floating inside the outer braid; and
wherein the tapered proximal end of the plug has angle of more than 30 degrees with respect to an axis that is perpendicular to the longitudinal axis.

8. The plug of claim 7, wherein the inner braid is located inside the outer braid.
